(19) United States
(12) Patent Application Publication
(10) Pub. No.: US 2026/0283649 A1
(43) Pub. Date: Sep. 24, 2026

(54) ROBOTIC AND NAVIGATED ROD BENDING

(71) Applicant: GLOBUS MEDICAL, INC., AUDUBON, PA (US)

(72) Inventors: Timothy James, Hatboro, PA (US); Neil R. Crawford, Chandler, AZ (US); George Yacoub, Lansdale, PA (US); Sarah Wolverton, Phoenixville, PA (US)

(21) Appl. No.: 19/629,131

(22) Filed: Mar. 26, 2026

Related U.S. Application Data
(63) Continuation of application No. 18/632,478, filed on Apr. 11, 2024.
Application No. 18/632,478 is a continuation-in-part of application No. 18/352,313, filed on Jul. 14, 2023.

(51) Int. Cl. A61B 17/70; A61B 17/00; A61B 34/20; A61B 34/32; A61B 46/10

(52) U.S. Cl. CPC A61B 17/7013; A61B 34/32; A61B 46/10; A61B 2017/00477; A61B 2034/2072

(57) ABSTRACT

Rod bending instruments, systems, and methods thereof are associated with robotic and navigated bending of a rod for spinal surgeries. A system for bending a spinal rod includes a rod bending assembly and an automatic or navigated feeding system. The rod bending assembly includes a bender box having a fixed coupling member and an actuated coupling member. A rod cutter is attachable to the fixed coupling member and a bending mandrel is attachable to the actuated coupling member, for example, over a sterile drape. The automatic or navigated feeding system is configured to feed a spinal rod into the rod bending assembly to bend and contour the spinal rod into a complex three-dimensional shape.

### CROSS-REFERENCE TO RELATED APPLICATION(S)

This patent application is a continuation application of U.S. Ser. No. 18/632,478, filed Apr. 11, 2024, which is a continuation-in-part of U.S. patent application Ser. No. 18/352,313, filed on Jul. 14, 2023, all of which are incorporated by reference herein in their entireties for all purposes.

### FIELD OF THE INVENTION

The present disclosure relates generally to instruments, systems, and methods for robotic and navigated orthopedic surgery in which powered mechanical components are present in the sterile field, and in particular to surgical operations which include procedures for bending of a rod in the context of spinal surgeries.

### BACKGROUND OF THE INVENTION

Surgical navigation has revolutionized spine surgery by allowing surgeons to accurately and repeatably place implant hardware with decreased intra-operative radiation and operative time as opposed to conventional surgical techniques. When screws are placed in these procedures, spinal rods are placed as the final step to achieve correction. Recently, there have been advances with patient specific pre-operative rods and assisted intra-operative bent rods but the vast majority of rods need to be manually bent to achieve the surgical goals.

Rod bending takes place at the end of the procedure, after screws and interbody implants are placed. Manual rod bending is a skill intensive task that may utilize a combination of French benders, table benders, and in situ benders to reach the desired correction. It can be challenging to match the curve on the left and right sides, high stresses can be placed on the rods during bending, and if a rod becomes damaged or unsterile during the surgery everything needs to be redone from scratch.

Assisted intra-operative bending started making improvements to manual rod bending but still has some major shortcomings and low adoption. Once the screws and interbody implants are placed, the surgeon may use a navigated probe to verify the location of each screw. The software may take that data to generate a point to point curve with bends at the screw heads. The plan may include axial positions, bend angles, and roll angles at each bend. This assumes that the bend is at each screw. If the user wants to smooth out the curve or account for desired correction, the user needs to teach the plan additional points.

Patient specific pre-operative rods may be built from a pre-operative plan, created with sufficient lead time to allow for the rods to be manufactured for the operation. This approach produces a contoured rod with minimal defects but requires a detailed pre-operative plan. Any intra-operative deviations to the plan require manual bending, which can stress and weaken the rod and defeats the purpose of a patient specific rod. As such, there exists a need for instruments and systems capable of addressing one or more of these limitations.

### SUMMARY OF THE INVENTION

To meet this and other needs, and in view of its purposes, the present application provides instruments, systems, and methods for robotic and navigated rod bending of a rod for spinal surgeries. In particular, the spinal rod may be contoured into a complex three-dimensional (3D) shape to match the patient's spine, align with and seat in screw heads fixed to the spine, and be deformable to achieve the desired correction when corrective forces are applied. The rod bender system may include a bender box, for example, attachable to a robot, with a bend mandrel and rod cutter assembly. The rod bender system may be controllable by an automated robot and/or via navigated assistance. Furthermore, an intra-operative rod bending system may be used to generate an intra-operative rod plan based on screw placement and/or user input. The rod plan may be used to produce patient specific rods meeting the planned alignment goals during the procedure.

According to one embodiment, a system for bending a spinal rod includes a rod bending assembly and an automatic or navigated feed system. The rod bending assembly includes a bender box and a bender assembly coupled to the bender box. The bender box includes a top plate having a fixed coupling member and an actuated coupling member. The bender assembly includes a rod cutter attachable to the fixed coupling member and a bending mandrel attachable to the actuated coupling member. The automatic or navigated feeding system is configured to feed a spinal rod into the rod bending assembly. The bender assembly is configured to bend and contour the spinal rod into a complex three dimensional shape.

The system may include one or more of the following features. The bending mandrel may include a roller and a cam base. The roller may be a vertically oriented cylinder with a radial groove configured to receive the spinal rod. The rod cutter may include a block attached to the fixed coupling member, a fixed plate attached to the block, and a moveable plate pivotally coupled to the fixed plate. The moveable plate may be pivotally coupled to the fixed plate via a pivot pin. The fixed plate and the moveable plate may define through openings, and when aligned, the fixed and moveable plates may be configured to receive the spinal rod therethrough. The moveable plate may include a lever arm with a handle configured to pivot the moveable plate. The automatic or navigated feeding system may be an automatic robot configured to feed and rotate the spinal rod. Alternatively, the automatic or navigated feeding system may be a navigated handle having a plurality of tracking markers attachable to one end of the spinal rod.

According to one embodiment, an automatic rod bender system includes a surgical robot and a rod bender assembly. The surgical robot may include a base having transport handles and including a computer, a robot arm electronically coupled to the computer and moveable based on commands processed by the computer, and an end-effector coupled to the arm. The rod bender assembly may be attachable to the transport handles of the surgical robot. The rod bender assembly may include a bender box having a fixed coupling member and an actuated coupling member. A rod cutter may be attachable to the fixed coupling member and a bending mandrel may be attachable to the actuated coupling member. The end-effector may be configured to hold one end of a spinal rod to automatically feed and rotate the rod through the rod bender assembly.

The automatic system may include one or more of the following features. The end-effector may include a passive palm joint permitting the end effector to rotate and a passive finger joint configured to allow the end effector to pivot, thereby allowing the spinal rod to freely rotate to any angle. The end-effector may include a clamp configured to attach to the arm of the robot, an inner coupling plate affixed to the clamp, a yoke rotatable about the inner coupling plate, a clevis assembly pivotably coupled to the yoke, and a collet attached to the clevis assembly and configured to secure the spinal rod. The yoke may include an outer ring with struts extending therefrom. The outer ring may define an inner stepped recess configured to receive a ledge on the inner coupling plate, thereby permitting the yoke to rotate with respect to the inner coupling plate. The clevis assembly may include a clevis pin connected to an outer member and a finger extending through the outer member. A thumb lever may extend perpendicularly from the finger, and the thumb lever may be rotatable between multiple slots in the outer member to allow a user to rotate the spinal rod. The collet may include a rotatable outer spindle and an inner collar divided into segments by a series of slits. When the outer spindle is rotated, the inner collar segments contract, thereby gripping the spinal rod.

According to one embodiment, a method of bending a spinal rod includes one or more of the following steps in any suitable order: (1) providing a robot having a base with transport handles and including a computer, a robot arm electronically coupled to the computer and moveable based on commands processed by the computer, and an end-effector coupled to the arm; (2) attaching a bender box to one transport handle of the robot, the bender box having a fixed coupling member and an actuated coupling member controllable by a power source and a data cable; (3) optionally, applying a sterile drape between the bender box and the bender assembly and/or over the robot; (4) attaching a bender assembly to the bender box by affixing a rod cutter to the fixed coupling member and affixing a bending mandrel to the actuated coupling member; (5) securing a spinal rod to the end-effector, for example, by rotating a collet; (6) automatically feeding the spinal rod through the bender assembly to bend and contour the spinal rod, for example, by sequentially feeding the spinal rod along its axis, rotating about its axis, and bending by rotating the bending mandrel, in sequence, until the rod is fully bent; and (7) automatically actuating the rod cutter to cut the spinal rod to length. If desired, an intra-operative rod plan may be developed based on screw placement and/or user input to produce patient specific rods meeting the planned alignment goals during the procedure. The custom spinal rod may be aligned with, seated within, and secured within screw heads affixed to the spine, and the spinal rod may be optionally reduced into position to achieve the desired correction of the spine.

Also provided are kits including surgical instruments of varying types, spinal rods, fasteners or anchors, k-wires, insertion tools, and other components for performing the procedure.

According to still other possible implementations, a system for bending a spinal rod comprises a rod bending assembly, an automatic or navigated feeding system configure to feed a spinal rod into the rod bending assembly, and a sterile barrier located between a bender box of the rod bending assembly and a bender assembly of such rod bending assembly. The sterile barrier extends sufficiently to define a non-sterile side associated with the bender box and a sterile side associated with the bender assembly. A non sterile coupling and a sterile coupling are interconnected by a mechanical connection through the sterile barrier. The mechanical connection may be in the form of a sterile floating coupling rotatably received in a coupling housing. The coupling housing, in turn, is secured to the sterile barrier and extends between opposite surfaces of such sterile barrier. As such, the floating sterile coupling transfers motion across the sterile barrier without corresponding movement of the sterile barrier.

In certain versions, the floating sterile coupling is releasably interconnected to opposing surfaces of the non-sterile coupling and the sterile coupling by mechanical engagement of opposing, keyed features located on such opposing surfaces.

In other versions, a plurality of floating sterile couplings is received in respective housings on the sterile barrier and interconnected to a corresponding plurality of non-sterile and sterile couplings. The plurality of floating couplings maybe located in a coupling housing sized to receive the floating couplings at predetermined locations corresponding to desired mechanical connections between non-sterile, power-driven components of a surgical or robotic system and the sterile side of such systems where a sterile cartridge or other sterile components acted upon by a robot arm or end effector are located.

According to further implementations, a system for bending a spinal rod may be programmed with computer executable instructions which make use of patient and other relevant data in generating a surgical plan, including rod bending. Suitable programming, when executed, may receive input, either from a user, such as a surgeon, or by accessing data from a relevant database, such input corresponding to proposed repositioning of vertebrae of the patient's spine to achieve a spinal correction. The proposed repositioning input may be used to generate a corresponding surgical or rod bending plan. The system may permit the user to, or itself may access a database so as to associate a set of screws with the proposed spinal correction. Suitable input or data is likewise accessed for at least one rod corresponding to a preliminary, proposed rod configuration related to the set of screws for the spinal correction. In response to receiving the rod input, suitable programming generates a preliminary rod-and-screw construct connecting the set of screws and the at least one rod to achieve the spinal repositioning which has been inputted.

In certain implementations, the foregoing system may include suitable programming for determining a rod-and-screw stiffness corresponding to the preliminary rod-and-screw construct. Such rod-and-screw stiffness may be determined as a function of parameters corresponding to the rod, that is, rod input, and parameters associated with the set of screws, such as screw angulation. Suitable programming may determine another factor, namely, a patient's spine stiffness factor which corresponds to a spinal counterforce associated with patient specific data. Such spine stiffness factor may thus affect the proposed spinal correction for the patient. Patient spine stiffness data which may be factored into the determination of the spine stiffness factor includes static forces generated by the patient's spinal connective tissues, such as those arising from ligaments, discs, and muscle. Additional patient stiffness data which may be factored into the determination of the spine stiffness factor may involve patient data such as age, gender, bone quality, radio graphic status of intervertebral discs, negative health factors, and, where appropriate, ethnicity.

In still other possible implementations, a system for bending a spinal rod may include suitable programming to factor in screw reachability and corresponding rod plans associated with reachability of a given set of screws proposed for a rod-and-screw construct or associated surgical plan. In certain exemplary embodiments, suitable programming processes screw angulation data and potentially other relevant screw data for a proposed set of screws to determine reachability or unreachability of this set of screws by at least one rod of a proposed rod plan. The computerized system may signal a determination of unreachability to the user in real time, and thereafter receive further user input substituting at least one screw in response to the unreachability determination to thereby update the proposed set of screws.

In response to the updated set of screws, suitable programming may repeat the determination of reachability or unreachability. Upon a determination of reachability for all screws proposed for the surgical or rod plans, the system may generate the resultant plan. In other versions of the foregoing, screw parameters and the determination of reachability may be factored into different proposed rod curvatures of the rod plan such that a set of potential rod plans is generated from which the user, such as a surgeon, may select one meeting certain desirable criteria.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows an automated robot with a rod bender system having a bending box and a bending assembly configured to bend and cut a spinal rod according to one embodiment;

FIG. 2 shows a bending box attached to the frame of the robot according to one embodiment;

FIG. 3 shows the bending box of FIG. 2 without the outer case according to one embodiment;

FIG. 4 shows the robot and bending box with a drape configured to maintain sterility according to one embodiment;

FIG. 5 shows a close-up view of a rod cutter and bend mandrel assembly attached to the bending box according to one embodiment;

FIGS. 6A-6B show top-down views of the robot and rod bender system with the robot arms locating the spinal rod in its outer-most feed position and its inner-most feed position, respectively, according to one embodiment;

FIGS. 7A-7B show perspective and cross-sectional views, respectively, of an end effector with a passive palm joint and a passive finger joint for controlling movement of the spinal rod relative to the rod bender system according to one embodiment;

FIGS. 8A-8C show the robot and rod bender system with the spinal rod in its further out rod position, with furthest clockwise rotation, and with furthest counter-clockwise rotation, respectively, according to one embodiment;

FIGS. 9A-9C show the robot and rod bender system with the spinal rod in its further in rod position, with furthest clockwise rotation, and with furthest counter-clockwise rotation, respectively, according to one embodiment;

FIG. 10 shows the robot automatically cutting the spinal rod according to one embodiment;

FIG. 11 shows a perspective view of an end effector with a motor according to one embodiment;

FIG. 12 shows a rod bender system with navigation assistance according to one embodiment;

FIG. 13 shows a close-up view of the navigated handle according to one embodiment;

FIG. 14 is a flowchart of a patient specific plan workflow according to one embodiment where the patient specific rod plan may be generated from points correlated to placed screw data;

FIG. 15 is a flowchart of a screwless rod plan workflow according to one embodiment where a generic rod plan may be generated from points not correlated to screw placement;

FIG. 16 is a flowchart of a bend sequence workflow according to one embodiment where bend points may be generated based on a final rod plan and fed into an automatic rod bending system;

FIG. 17 is a flowchart of an overall system workflow according to one embodiment;

FIG. 18 shows a system for navigated point capture according to one embodiment with an instrument capable of relaying point data back to the planning system;

FIG. 19 shows a differential rod feeding system configured to control linear translation and roll angle of a spinal rod according to one embodiment;

FIG. 20 shows mirrored angled rollers configured for feeding and rotating the spinal rod according to one embodiment;

FIG. 21 shows a pair of mirrored blocks configured to control translation and rotation of the spinal rod according to one embodiment;

FIG. 22 shows a radially adjustable rod feeding system with rollers mounted on slide blocks to accommodate multiple rod diameters according to one embodiment;

FIG. 23 shows one possible implementation of a graphical user interface for the automated robot with rod bending system of FIGS. 1-4;

FIG. 24 shows the system of FIG. 23 with suitable, computer programming, and a schematic of certain, exemplary workflows, which, when executed, process certain data in generating surgical or rod plans;

FIG. 25 shows other exemplary workflows of embodiments of the programmed system disclosed herein;

FIG. 26 show screw types associated with the programmed system disclosed herein;

FIGS. 27-28 show a schematic of one suitable graphical user interface associated with still further robotic systems for bending rods;

FIG. 29 shows one implementation of a workflow executable by suitable computer instructions factoring screw reachability when generating rod plans associated with the systems disclosed herein;

FIG. 30 shows an isometric view of one implementation of a non-sterile motion table and sterile cartridge of the presently disclosed systems;

FIG. 31 shows an exploded, isometric view of one implementation of a sterile, floating coupling for a sterile barrier of the presently disclosed systems;

FIG. 32 shows a sectional, elevation view of another implementation of the sterile, floating coupling of FIG. 31;

FIG. 33 shows an exploded, isometric view of the implementation of the sterile, floating coupling of FIG. 32;

FIG. 34 shows another exploded, isometric view of certain components of the sterile, floating coupling of FIG. 33;

FIG. 35 shows an exploded, isometric view of certain components of another embodiment of a sterile, floating coupling;

FIG. 36 shows a top plan view of another embodiment of a coupling housing for use with a plurality of sterile floating couplings; and '

FIG. 37 shows an exploded perspective view of the sterile floating coupling embodiment of FIG. 36.

### DETAILED DESCRIPTION OF THE INVENTION

Embodiments of the disclosure are generally directed to instruments, systems, and methods for robotic and navigated rod bending of a rod for spinal surgeries. In particular, a rod bender system may be used to contour the spinal rod into a complex three-dimensional (3D) shape to match the patient's spine, align with and seat in screw heads fixed to the spine, and be deformable to achieve the desired correction when corrective forces are applied. The rod bender system may include a bender box with a bend mandrel and rod cutter assembly attachable thereto. The rod bender system may be controllable by an automated robot, via navigated surgical assistance, or by another suitable rod feeding system.

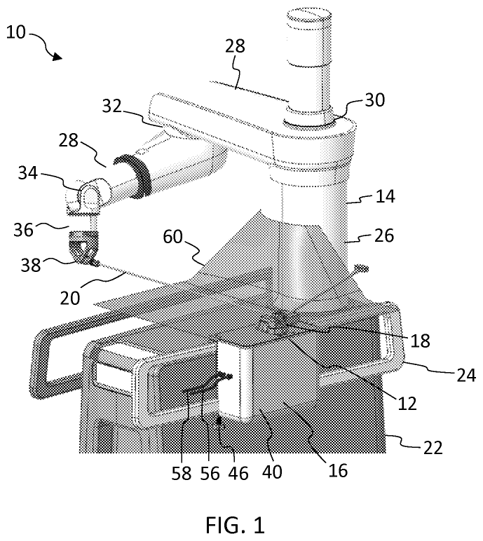

Referring now to FIG. 1, an automated rod bender system 10 is shown according to one embodiment. In this embodiment, robotic automated bending 10 includes a rod bending assembly 12 configured to be controlled by a surgical robot and/or navigation system 14. The rod bending assembly 12 may include a bender box 16 attachable to the robot 14 and a bender assembly 18 coupled to the bender box 16. The bender assembly 18 is configured to bend and contour a spinal rod 20 into a complex and custom three-dimensional shape.

Spinal rods 20 are used in surgical procedures to stabilize the spine, correct deformities, and maintain proper alignment of the spine. The spinal rod 20 may be an elongated shaft having a generally cylindrical outer body. The rod 20 may be made from materials, such as titanium or stainless steel that have high tensile strength and can withstand forces and stresses placed on the spine. It will be appreciated that the spinal rod 20 may also have other cross-sectional shapes, such as oval, rectangular, or flattened surfaces or may be made from other suitable materials. The length and diameter of the rod 20 can vary depending on the surgeon's preference and the patient's anatomy. During the surgery, the surgeon may need to bend the rod 20 to match the patient's spinal curvature, to align with new or existing hardware, such as screw heads or tulip heads, and/or to achieve the desired correction when corrective forces are applied to the rod 20. Examples of bone fasteners, other implants, and rod constructs are described in more detail, for example, in U.S. Pat. No. 10,603,081, which is incorporated by reference herein in its entirety for all purposes. The bender assembly 18 may be used to bend and/or shape the rod 20 to achieve the desired curvature and alignment.

In one embodiment, the surgical robot and/or navigation system 14 may be used to automate the rod bending process. For example, the robot 14 may be used for feeding, rotating, and/or cutting the rod 20. The robot 14 may be a serial arm manipulator including, for example, a base 22 on wheels having transport rails or handles 24 and containing one or more computers having a processor, programming, and/or memory; an optional display, monitor, and/or wireless tablet (not shown) electronically or wirelessly connected to the computer; a vertical column 26 extending from the base 22 and supporting one or more moveable robot arms 28 at a shoulder joint 30 controlled by at least one motor based on commands processed by the computer; and an end-effector 38 coupled to a palm 36 at a wrist joint 34. The end-effector 38 is configured to securely hold one end of the spinal rod 20 to move and orient the rod 20 in three-dimensional space.

The surgical robot and/or navigation system 14 may also utilize tracking markers and a camera (not shown), for example, positioned on a camera stand to move, orient, and support the camera in a desired position. The camera may include any suitable camera, such as one or more infrared cameras (e.g., bifocal or stereophotogrammetric cameras), able to identify and track, for example, active and passive tracking markers in a given measurement volume. The system may further include 2D & 3D imaging software that allows for pre-operative and intra-operative planning, navigation, and guidance. Further examples of surgical robotic and/or navigation systems can be found, for example, in U.S. Pat. Nos. 10,675,094 and 9,782,229, which are incorporated by reference herein in their entireties for all purposes.

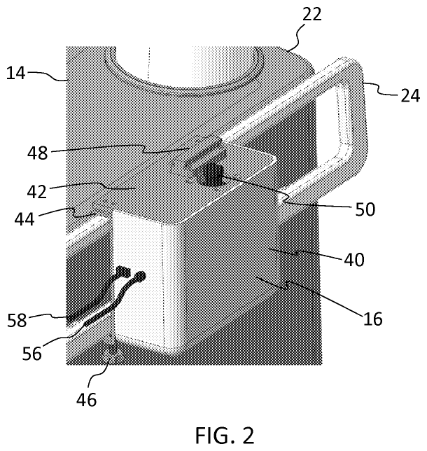

With further reference to FIG. 2, the bender box 16 is shown in more detail. The bender box 16 includes an outer casing or housing 40 with a top plate 42. The bender box 16 is configured to attach to the frame, rail, or handle 24 of the robot 14, with a temporary or adjustable connection. For example, the bender box 16 may be temporarily connected to the handle 24 of the robot 14 by an upper hook 44 and a lower clamping knob 46, which may be manually tightened by the user. The hook 44 may include an L-shaped body for hanging over the top edge of the robot handle 24. The upper hook 44 may extend the entire length of the bender box 16, for example. The clamping knob 46 may include a rotatable knob attached to a threaded rod or stud with a lower clamping portion configured to grip the bottom edge of the robot handle 24. For example, a pair of clamping knobs 46 may be provided on either side of the bender box 16. When the knob 46 is rotated, the clamping mechanism is tightened to securely hold the bender box 16 to the robot 14. Although a specific configuration for attaching the bender box 16 to the robot 14 is shown, it will be appreciated that any suitable type of fastening or attachment system may be used to temporarily and securely hold or connect the bender box 16 to the robot 14. It will also be appreciated that although shown attached to robot 14, the bender box 16 may be attached to another appropriate location, such as the patient bed, operating room table, cart, etc.

The top plate 42 of the bending box 16 has a fixed sterile coupling member 48 and an actuated sterile coupling member 50. The fixed coupling member 48 may include a rail or track protruding from an upper surface of the top plate 42. The rail 48 may have a T-shaped cross-section with a horizontal base and a central vertical bracket or another suitable configuration. The fixed coupling member 48 allows for the insertion and attachment of the bender assembly 18 to the top of the bender box 16. The actuated sterile coupling member 50 may be moveable and configured to control the rod bending interface. The actuated sterile coupling member 50 may include a switch or lever that can be tilted, pivoted, rotated or moved in different directions. The lever 50 may project upwardly through the top plate 42 to engage the bender assembly 18.

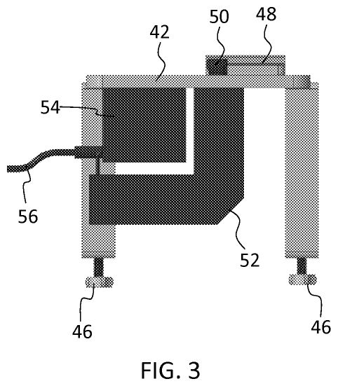

With further emphasis on FIG. 3 showing the bender box 16 without the case 40, the actuated coupling member 50 is controlled by one or more electric motors 52, such as servomotors. The electric motors 52 are supplied with power and control signals from an electronics package 54, which houses power regulators and motor controllers. The electronics package 54 may be connected to and controlled by the robot or navigation platform 14 via a data cable 56 and connected to a power source via a power cable 58. It will be appreciated that the actuated coupling member 50 may be controllable via data cable 56 connected to any computer or processor and power source 58 or any other appropriate substructure or configuration.

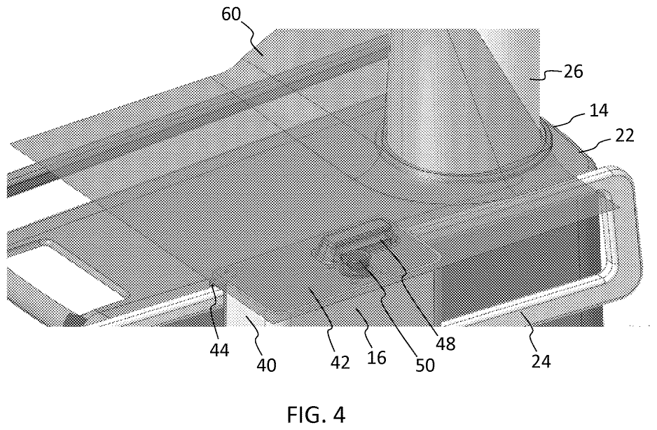

Turning now to FIG. 4, a drape 60 is placed over the bender box 16 and any surfaces the spinal rod 20 passes over to maintain sterility. The drape 60 may be an extended portion of the drape used to cover the robot 14 or a separate drape. The drape 60 covers the fixed and actuated sterile coupling members 48, 50. Sterile components, such as bender assembly 18 are then attached over the drape 60 onto the sterile coupling members 48, 50 and locked thereto. Slack left in the drape 60 allows the bending mandrel 64 to rotate up to a maximum angle and bend the spinal rod 20 without tearing the drape 60.

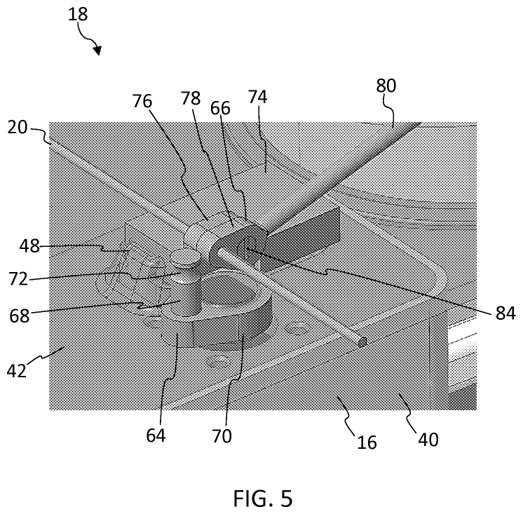

Turning now to FIG. 5, a close-up view of the bender assembly 18 is shown. The bender assembly 18 includes a bending mandrel 64 configured to bend the spinal rod 20 and a rod cutter 66 configured to cut the spinal rod 20 to length. The bending mandrel 64 includes a roller 68 and a cam base 70. The roller 68 may include a vertically oriented cylinder with an upper radial groove or recess 72 configured to receive the spinal rod 20 therein. As the bending mandrel 64 rotates, the radial recess 72 is configured to engage and bend the spinal rod 20 without notching or otherwise damaging the spinal rod 20. The roller 68 is attached to the cam base 70, which may be fixed or rotatable relative to the cam base 70. The cam base 70 is configured to couple to the actuated coupling member 50. When the actuated coupling member 50 moves or rotates, the cam base 70 follows the movement of the actuated coupling member 50. In this manner, the actuated coupling member 50 directly moves or rotates the bending mandrel 64 to engage and bend the spinal rod 20. The cam base 70 may have an asymmetric profile, such as pear shape, wedge shape, or irregularly shape. In one embodiment, the cam base 70 is pear shaped with a circular arc having an axis of rotation at one end and the roller 68 extending upward from the opposite narrowed end. The spinal rod 20 is bent by sequentially feeding the rod 20 along its axis, rotating the rod 20 about its axis, and bending the rod 20 by rotating the bending mandrel 64. This sequence is repeated until the rod 20 is fully bent to the desired custom shape. In addition, the bending mandrel 64 may apply slight pressure to the spinal rod 20 to act as a brake and hold the rod's orientation temporarily between moves.

The rod cutter 66 includes a block 74 attachable to the fixed coupling member 48, a fixed plate 76 attached to the block 74 and configured to hold rod 20 therein, and a moveable plate 78 configured to hold and cut the rod 20 via movement of lever arm 80. The block 74 may be a rectangular cube having a groove or recess on its underside configured to interface with the fixed coupling member 48. For example, the bottom of the block 74 may define a recess configured to accept the t-shaped rail of the fixed coupling member 48. The block 74 may be rigidly secured to the fixed coupling member 48 via a dovetail joint, mortise and tenon joint, lap joint, etc.

The fixed plate 76 may be attached to a top surface of the block 74. The fixed plate 76 defines a through opening sized and dimensioned to receive the spinal rod 20. The opening may be oriented to hold the spinal rod 20 along a horizontal plane. The moveable plate 78 is positioned next to the fixed plate 76, for example, parallel to one another. The moveable plate 78 may be secured to the fixed plate 76 with a pivot pin 84 or the like. The moveable plate 78 also has a through opening sized and dimensioned to receive the spinal rod 20 and when in a neutral position, the through opening of the moveable plate 78 is aligned with the through opening of the fixed plate 76, thereby permitting the spinal rod 20 to pass therethrough.

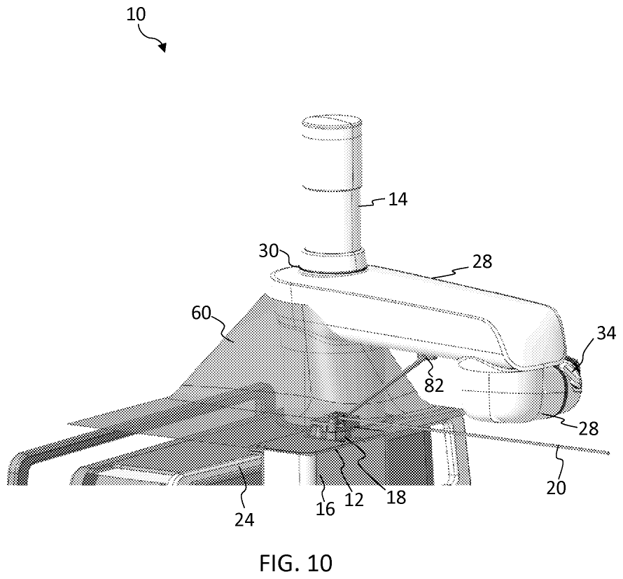

The lever arm 80 extends off the back end of the moveable plate 78 adjacent to the pin 84 and opposite to the rod through opening. The lever arm 80 may include a shaft that is attached to or integral with the moveable plate 78. The free end of the lever arm 80 may have an enlarged handle 82. The pivot pin 84 connecting plates 76, 78 has a pivot axis configured to rotate moveable plate 78 about its pivot axis when the lever arm 80 is moved up or down. For example, after bending to its desired shape, the spinal rod 20 may be cut by applying a downward force onto the lever arm 80. The lever arm 80 pivots moveable plate 78 and shears the rod 20 against the fixed plate 76, which is rigidly coupled to the fixed sterile coupling member 48. Thus, when the lever arm 80 is forced downward, the moveable plate 78 causes a shear force to cut the rod 20 to its desired length. The lever arm 80 may be operated manually by the user pressing downward on handle 82 or the force may be applied automatically by the robot arm 28, for example, as shown in FIG. 10.

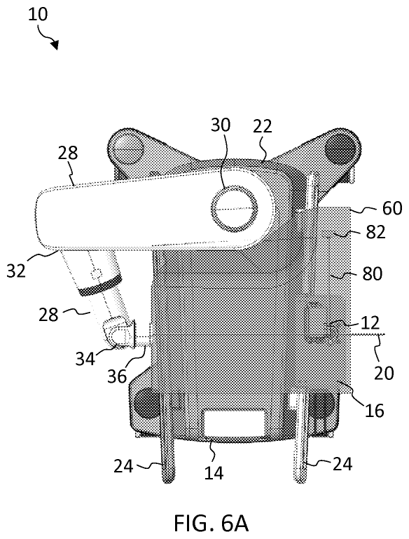

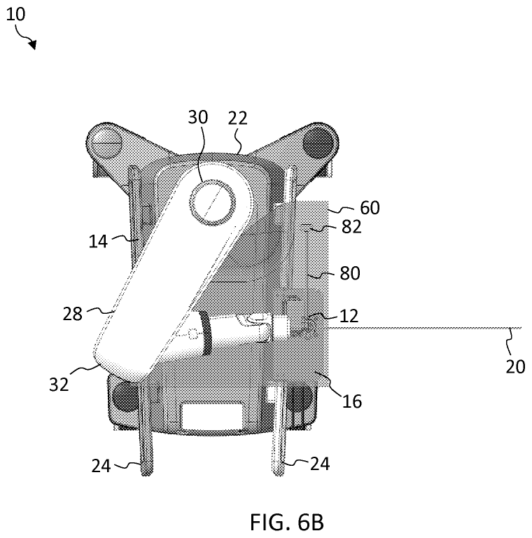

FIGS. 6A-6B show top-down views of the robot 14 and rod bender assembly 12 with the robot arms 28 locating the spinal rod 20 in its outer-most feed position and its inner-most feed position, respectively. In this embodiment, the robot 14 acts as a serial arm manipulator for feeding, rotating, and cutting the rod 20 to automate the rod bending process. The end effector 38 is attached to the palm 36 of the serial arm manipulator. The end effector 38 may be coupled to the palm 36 across the sterile drape 60 via a sterile coupler. The end effector 38 is coupled to the spinal rod 20 so that the rod 20 is controlled by the motion of the serial arm manipulator 14. The bend box 16 is attached to the rails 24 at the base 22 of the serial arm manipulator. The robotic bending setup is shown with the long rod 20 rigidly attached to the end effector 38. In FIG. 6A, at the furthest rod out position, the wrist 34 of the robot 14 has reached the limit of its range of motion, leaving a length of the rod 20 which cannot be bent. In FIG. 6B, at the furthest rod in position, the shoulder 30 of the robot 14 has reached its limit and prevents the rod 20 from fully entering the bend mechanism of the bending assembly 12, leaving unbent rod on the other end of the rod 20. Additionally, in this set-up, the robot 14 cannot control rotation of the rod 20 independent of translation as all of its degrees of freedom are fully constrained by positioning the spinal rod 20.

As such, various end effector designs may be offered to provide additional passive or active joints. Such joints may provide additional degrees of freedom so that the spinal rod 20 may be fed and rotated along its entire length and all desired bends are within the reachable space of the serial arm manipulator 14. Alternatively, additional joints may be added to the serial arm manipulator itself to increase its reachability to feed and rotate rod 20.

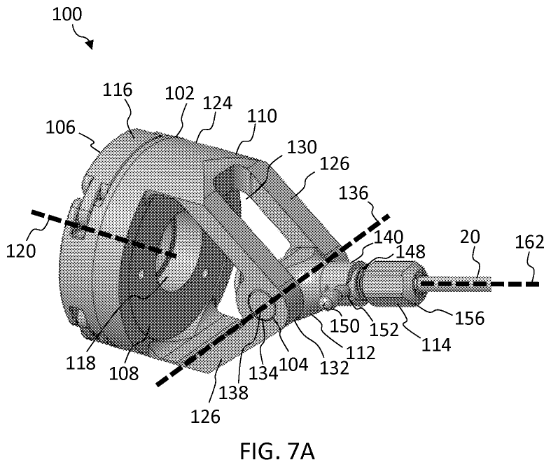

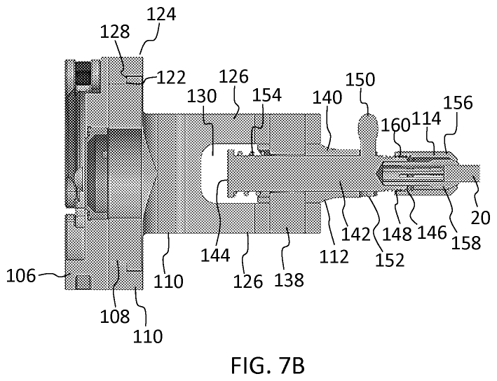

Turning now to FIGS. 7A-7B, an end effector 100 is shown according to one embodiment with passive joints 102, 104 configured to provide additional degrees of freedom to the rod 20. In this embodiment, end effector 100 includes a passive palm joint 102 permitting the end effector 100 to rotate and a passive finger joint 104 configured to allow the end effector 100 to pivot, thereby allowing the spinal rod 20 to freely rotate to any angle. The end effector 100 includes a sterile coupler or clamp 106 configured to attach to the palm 36 of the robot 14, an inner coupling plate 108 rigidly affixed to the clamp 106, a yoke 110 rotatable about the inner coupling plate 108, a clevis assembly 112 pivotably coupled to the yoke 110, and a collet 114 attached to the clevis assembly 112 and configured to secure the spinal rod 20.

The sterile coupler or clamp 106 is configured to attach to the palm 36 of the robot 14. The clamp 106 may mechanically interface with the end of palm 36 through one or more couplings, such as a magnetic kinematic mount. The clamp 106 may include a ring-like body with balls attracted by magnets in the palm 36 and a hinged clamp handle 116 for securing the end effector 100 to the palm 36. Examples of attachment mechanisms for an end effector to the robot arm is described in further detail in U.S. Publication No. 2017/0258535, which is incorporated by reference in its entirety for all purposes.

The inner coupling plate 108 is affixed to the clamp 106. The coupling plate 108 may form an inner ring with a central through opening 118. The clamp 106 and coupling plate 108 are aligned with the palm 36 of the robot 14 along central axis 120. The coupling plate 108 may be bolted or otherwise rigidly secure to the sterile coupler 106. As best seen in FIG. 7B, the coupling plate 108 may define a ledge 122 configured to mate with yoke 110, thereby allowing the yoke 110 to rotate about the coupling plate 108. This creates the passive palm joint 102, which allows the yoke 110 to rotate freely about the palm's central axis 120.

The yoke 110 includes an outer ring 124 and struts 126 extending therefrom. The outer ring 124 of yoke 110 is rotatably mated with the inner coupling plate 108. The outer ring 124 defines an inner stepped recess 128 configured to receive the ledge 122 of the inner coupling plate 108, thereby permitting the yoke 110 to rotate with respect to the inner coupling plate 108 about axis 120. The struts 126 may include two pairs of angled struts 126 defining an opening 130 therebetween. Upper and lower respective struts 126 may each connect at a distal end 132 of the yoke 110, thereby forming a triangular shape when viewed from the side. The distal end 132 of yoke 110 may define through openings 134 configured to receive clevis 112.

The clevis assembly 112 is configured to pivot about clevis axis 136, thereby permitting rotation of the clevis assembly 112 relative to the yoke 110. The clevis assembly 112 includes a clevis pin 138 attached to an outer member 140 and an inner member or finger 142 extending through the outer member 140. The entire clevis assembly 112 pivots in the end of the yoke 110. The clevis pin 138 may be a pin or bolt receivable through the openings 134 in the yoke 110. The clevis pin 138 defines clevis axis 136. As the clevis assembly 112 pivots about clevis axis 136, this forms the passive finger joint 104.

As best seen in FIG. 7B, the inner finger 142 of the clevis assembly 112 extends from a proximal end 144 to a distal end 146 configured to attach to the collet 114. The finger 142 may define a shaft having exterior threads 148 at its distal end 146 configured to interface with corresponding threads 160 inside the collet 114. A thumb lever 150 may extend from the finger 142. For example, the thumb lever 150 may be oriented perpendicular to the finger 142. The thumb lever 150 may be rotated between multiple receptacles or slots 152 in the face of the outer member 140 to allow the user to rotate the spinal rod 20 about its axis 162. For example, the thumb lever 150 may be translated out of one receptacle or slot 152 and rotated into another slot 152 in the clevis 112 when extra range of motion is needed. A spring 154 located at the proximal end 144 of the finger 142 retains the thumb lever 152 within the slot 152 and prevents inadvertent rotation when not actuated by the user.

The collet 114 is used to rigidly clamp the spinal rod 20 to the end effector 100. The collet 114 may include a rotatable outer spindle 156 and inner collar 158 extending therethrough. The outer spindle 156 may have an outer surface configured to provide an enhanced grip for tightening or loosening, for example, with flats, knurls, ridges, etc. The inner collar 158 may be divided into segments by a series of slits running longitudinally. The spinal rod 20 is receivable within the distal end of the inner collar segments 158. As the outer spindle 156 is rotated or tightened, the inner collar segments 158 contract, thereby gripping the rod 20 securely and tightly. The proximal end of the collet 114 may include one or more inner threads 160 configured to interface with corresponding exterior threads 148 on the finger 142, thereby securing the collet 114 to the end of clevis assembly 112. When the spinal rod 20 is attached to the collet 114, the rod axis 162 is coaxial with the collet 114 and the inner finger 142. The passive palm joint 102 rotates freely about the central palm axis 120 and the passive finger joint 104 rotates freely about the clevis axis 136, thereby allowing the end effector 100 to freely rotate to any angle with respect to the rod's axis 162.

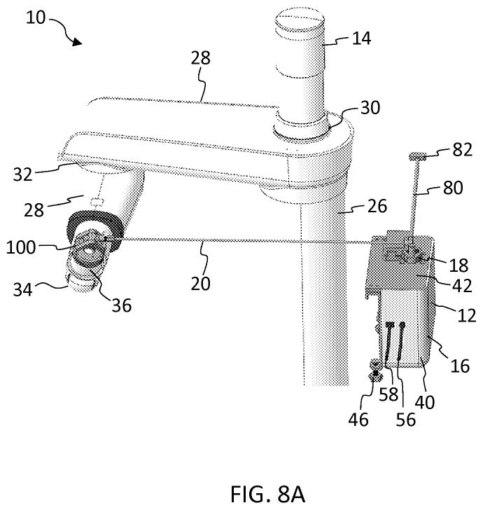

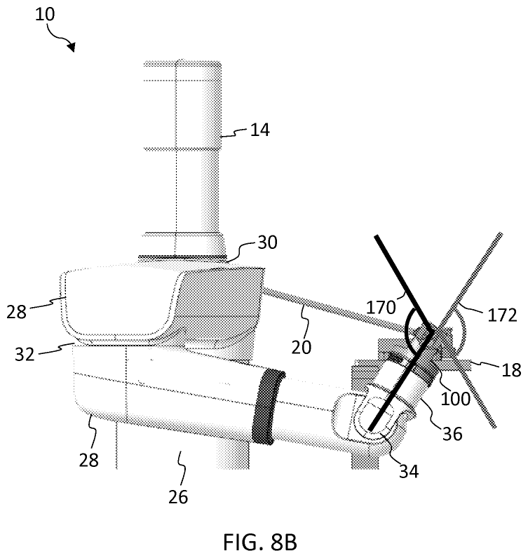

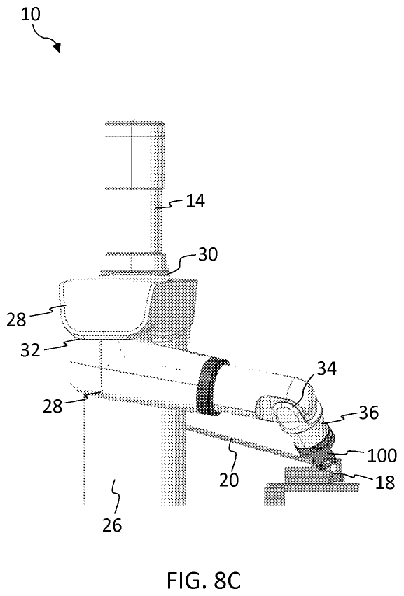

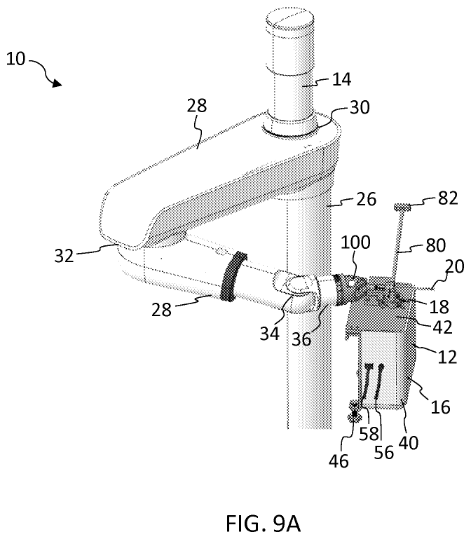

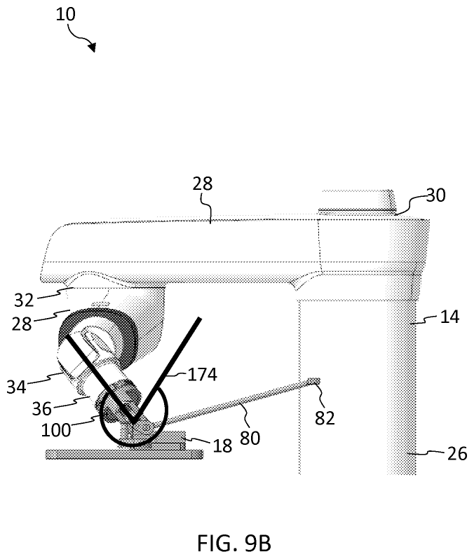

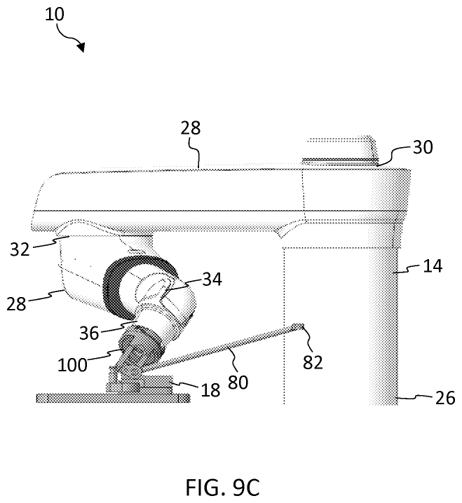

With further emphasis on FIGS. 8A-8C and 9A-9C, the two passive joints 102, 104 partially unconstrain two degrees of freedom of the serial arm manipulator 14. These joints 102, 104 increase the distance the robot 14 may feed the rod 20 enabling the arm 28 to rotate the rod 20 about its axis 162 independent of the distance the rod 20 has been fed. FIG. 8A shows the robot arm 28 at its furthest rod out position. At this furthest out position, the spinal rod 20 may be rotated within the limits of range of motion of the wrist 34 of the robot 14. If additional rotation is needed, the user may rotate the thumb lever 150 to another position to shift the wrist's range of motion to a different portion of the rod 20 and increase rotational reachability. FIG. 8B shows the furthest out rod position with the furthest clockwise rotation and two positions 170, 172 resulting in one area of additional rotational reachability 172. FIG. 8C shows the furthest out rod position with the furthest counter-clockwise rotation. FIG. 9A shows the robot 14 with the furthest in rod position. FIG. 9B shows the furthest in rod position with the furthest clockwise rotation. In the furthest in position, the rotation reachability 174 is limited by the range of motion of the elbow 32. The user may similarly flip the thumb lever 150 to increase the system's reachability should it be needed. FIG. 9C shows the furthest in rod position with the furthest counter-clockwise rotation. Other positions may provide additional reachability for rod 20.

Turning now to FIG. 10, the spinal rod 20 may be automatically cut to length by the robot 14. After contouring and bending the rod 20 to its desired shape, the spinal rod 20 may be disconnected from the end effector 38, 100. In one embodiment, the upper arm 28 of the robot 14 is lowered onto the handle 82 of lever arm 80 of the rod cutter 66 to cut the rod 20 to length. It will be appreciated that the robot 14 may be otherwise configured to move lever arm 80 or cut spinal rod 20 in another suitable manner.

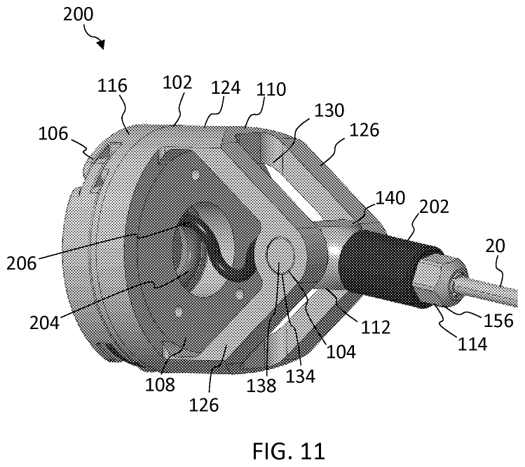

Turning now to FIG. 11, an electronic version of end effector 200 having passive palm and finger joints 102, 104 is shown according to another embodiment. End effector 200 is similar to end effector 100 except the clevis assembly is partially replaced with a motorized collet. In this embodiment, an additional sterilizable motor 202 is placed in line with the collet 114 of the end effector 200. The motor 202, such as an induction motor, brushless motor, servomotor, or the like may be used to actively control the collet 114 and rotation of the spinal rod 20 about its axis 162. The motor 202 may be powered and controlled by wireless power transmitted from the palm 36 of the robot 14, across the sterile drape 60, and picked up by an inductive coil 204 at the base of the end effector 200. A power cord 206 may transfer the power from the inductive coil 204 to the motor 202. Although a wireless power configuration is shown, it will be appreciated that the motor 202 may be battery operated or another suitable power source may be used. The powered end effector 200 enables direct control over rotation of the spinal rod 20 and full rotational reachability without intervention by the user.

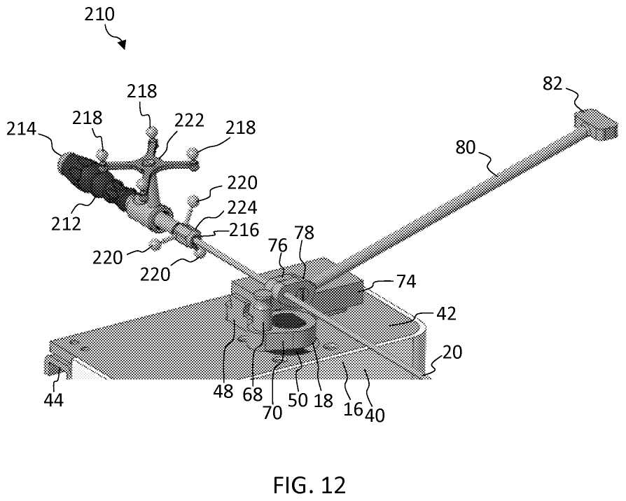

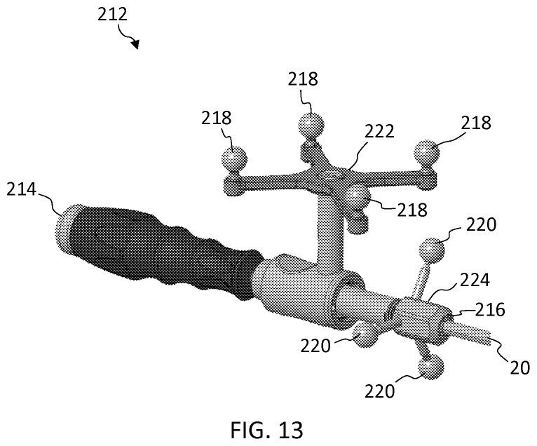

Turning now to FIGS. 12 and 13, a navigated rod bending system 210 is shown according to one embodiment. In this embodiment, the spinal rod 20 is fed and rotated manually by the user to the rod bending assembly 12. The spinal rod 20 is coupled to a navigated handle 212 having a handle grip and shaft extending along a central tool axis from a proximal end 214 to a distal end 216. The navigated handle 212 includes a plurality of tracking markers 218, 220 viewable and trackable by a navigation system, such as robot 14.

Infrared signal based position recognition systems may use passive and/or active sensors or markers 218, 220 for tracking the objects. For passive sensors or markers 218, 220, objects to be tracked may include passive sensors, such as reflective spherical balls or discs, which are positioned at strategic locations on the object to be tracked. Infrared transmitters transmit a signal, and the reflective marker 218, 220 reflect the signal to aid in determining the position of the object in 3D. For active sensors or markers, the objects to be tracked include active infrared transmitters, such as light emitting diodes (LEDs), and generate their own infrared signals for 3D detection.

In one embodiment, the trackable markers 218, 220 may include radiopaque or optical markers or fiducials. The markers 218, 220 may be suitably shaped, including spherical, spheroid, disc, cylindrical, cube, cuboid, or the like. In one embodiment, the markers 218, 220 coupled to instrument 210 comprise passive reflective fiducial spheres for navigation tracking. A first set of tracking markers 218 may be attached to a navigation array 222. The navigation array 222 is not rotationally constrained to the handle 212 in order to register translation of the spinal rod 20. A second set of set of markers 220 may be attached to the handle 212 to register rotation of the spinal rod 20. For example, multiple stray markers 220 may be attached with posts to the collet 224. The multiple stray markers 220 may be used to monitor rotation of the spinal rod 20 since a single marker could be obscured behind the rod 20 or apparatus during operation. Alternatively, machine vision may be employed to track the instrument 212 without any markers.

The spinal rod 20 may be rigidly attached to the handle 212, for example, by collet 224 at its distal end 216. The collet 224 may be similar to collet 114 and is configured to rigidly clamp the rod 20 to the handle 212. The rod bending assembly 12 may be set-up in a manner similar to that described for automatic system 10. In particular, the user may attach the bender box 16 to the frame 24 of the robot 14, patient bed, operating room table, cart, or the like. The user may apply a sterile drape 60 before attaching the bending mandrel 64 and rod cutter 66 to the bender box 16. While positioning and moving the spinal rod 20, translation and rotation of the tracking array 222 may be measured by the navigation system 14, which guides the user to feed and rotate the rod 20 to a designated position at bending mandrel 64. Once the desired position is achieved, the bender box 16 executes the bend as described with respect to system 10. Once the rod 20 is fully bent and contoured, the user manually pushes down on the handle 82 of lever arm 80 to cut the spinal rod 20 to the desired length. In this manner, the spinal rod 20 is fed and rotated by the user with navigated assistance to achieve the desired rod bending and contouring.

The advantages of robotic or navigation-assisted rod bending systems may include one or more of the following: (1) the ability to design the rod in software and fabricate the rod accurately; (2) decrease the likelihood of notching and yielding of the spinal rod induced by manual bending methods; and (3) decrease surgeon fatigue due to strenuous manual bending and cutting operations. The robotic bending system automates the bending process, allowing the surgeon to perform other operative tasks while the rod is bent. The bender assembly may integrate with existing robot systems used for pedicle screw placement in the operating room. Also, the system takes advantage of existing serial arm manipulator systems in order to feed and rotate the rod instead of additional complex mechanisms which must maintain sterility. The navigation-assisted rod bending system allows a user who does not use a robot to achieve precise control over feeding and rotating the spinal rod during the bending process.

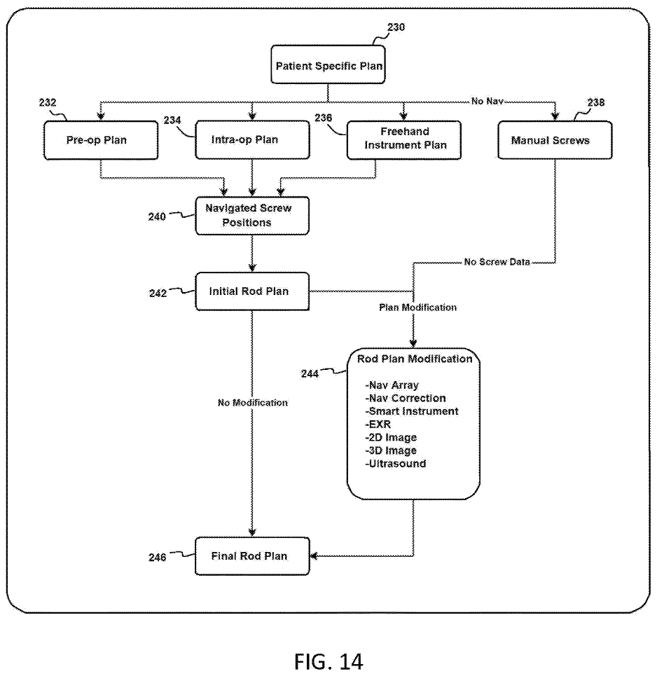

Turning now to FIGS. 14-17, rod bending workflows are described according to various embodiments. Specifically, an intra-operative rod bending system is configured to generate a rod plan based on screw placement and/or user input. The rod plan may be used to produce patient specific rods meeting the planned alignment goals and minimizing hardware failure.

According to one embodiment, an intra-operative navigation system is configured for capturing screw location data and generating points in 3D space. A navigated instrument is capable of being accurately tracked by the navigation system, attaching to the head of a placed screw, and indicating position data and trajectory to the navigation system. Pre-operative and intra-operative planning software is configured for generating a 3D curve incorporating multiple points, pre-planned or captured intra-operatively, and adjusting the 3D curve to produce desired correction. The intra-operative automatic rod bender is configured for bending rods in 3D space to a planned curve, bending rods of multiple diameters, bending rods of any clinically relevant length, trimming the rod to the desired length, and/or maintaining the sterility of the rod through the process.

With further emphasis on FIG. 14, a patient specific plan workflow 230 is shown according to one embodiment. The patient specific rod plan 230 may be generated from points correlated to placed screw data. The plan 230 may follow multiple methods including: a pre-operative plan 232, an intra-operative plan 234, a free-hand instrument plan 236 or no navigation for manual screws 238. For example, the screw data may be captured by pre-operatively planned screw trajectories, intra-operatively planned screw trajectories, intra-operatively saved navigated screw trajectories, and/or intra-operatively verified manual screw trajectories. Flowing from the pre-operative plan 232, the intra-operative plan 234, or the freehand instrument plan 236, the navigated screw positions 240 are determined and an initial rod plan 242 is developed. Once the initial rod plan 242 is generated or no screw data is obtained from manual screws 238, the user can begin or update the plan modification 244. For example, the rod plan modification 244 may include navigated verification array, navigated correction instruments, smart instrument data, navigated anatomy tracking (EXR/EVision), 2D anterior/posterior and lateral images, 3D computerized tomography (CT) scan or other images, and/or ultrasound. The initial rod plan 242 without modifications or the modified rod plan 244 results in a final rod plan 246 for bending and contouring the rod spinal 20.

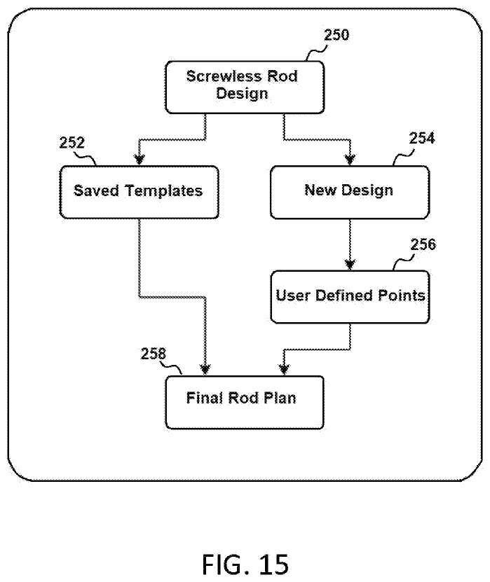

FIG. 15 shows a screwless rod workflow 250. The screwless rod design 250 may include saved templates 252 or a new design 254 obtain from user defined points 256, which lead to a final rod plan 258. The generic rod plan may be generated from points not correlated to screw placement. For example, the user inputs the point data in 3D space 256 or loads saved templates 252 to generate the final rod plan 258.

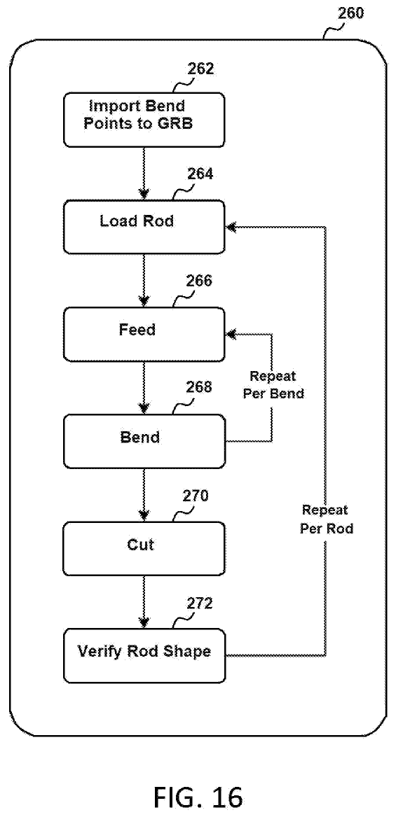

FIG. 16 shows a bend sequence workflow 260. The bend points may be generated based on a final rod plan and fed into an automatic rod bending mechanism. In this workflow 260, the steps may include: (1) import bend points in the rod bender system 262; (2) load the spinal rod 264; (3) feed the spinal rod 266; (4) bend the spinal rod 268 including repeating steps (3) and (4) per bend as necessary for the desired bending; (5) cut the rod to length 270; and (6) verify the rod shape 272. Steps (2) through (5) may be repeated per rod if necessary.

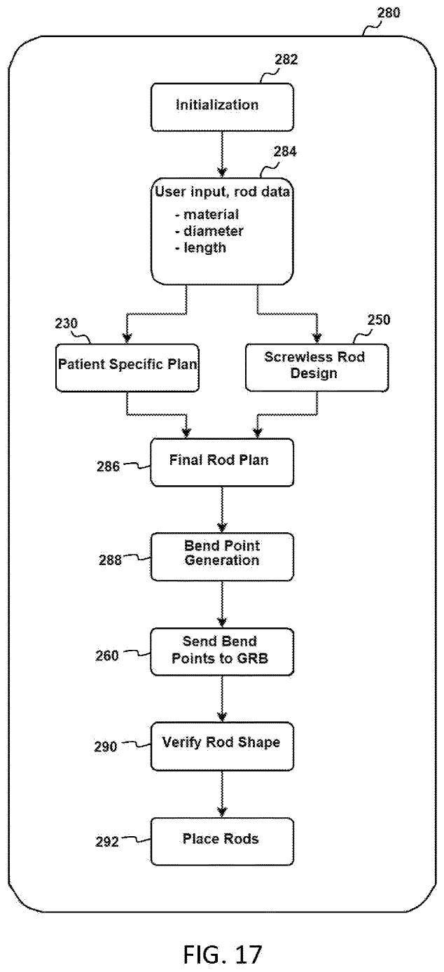

FIG. 17 shows an overall system workflow 280, which incorporates the workflows described above. For the overall rod bending workflow 280, the steps may include: (1) initialization 282 of the rod bender system, robot, navigation, etc.; (2), user input 284 of the rod data, such as material, diameter, and length; (3) choosing the patient's specific plan 230 or the screwless rod design 250; (4) outputting the final rod plan 286; (5) generating the bend points 288; (6) sending the bend points to the rod bender system 260; (7) verifying the rod shape 290; and (8) placing the spinal rods 20 into the patient 292.

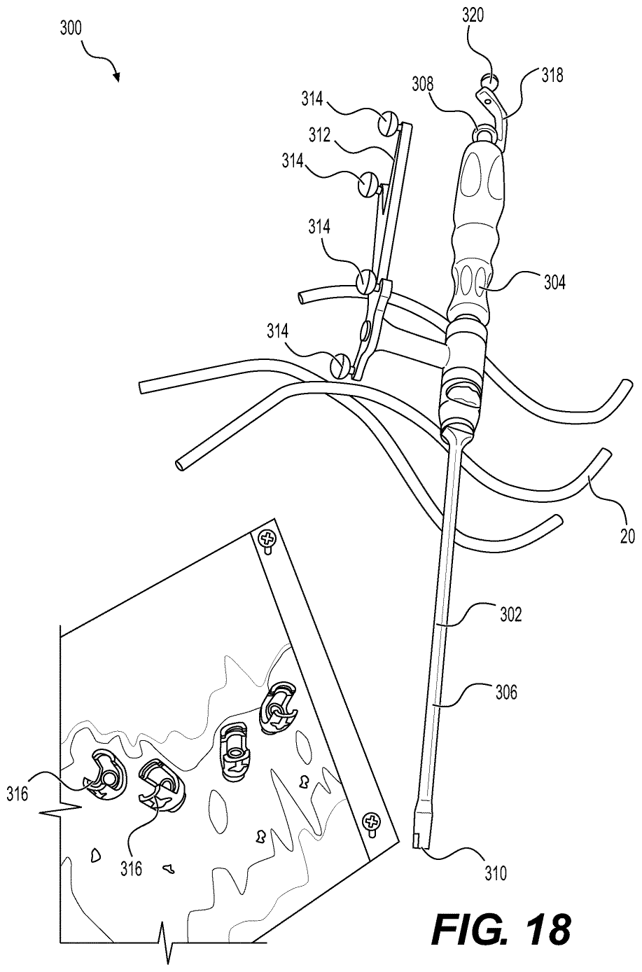

Turning now to FIG. 18, a navigated system 300 for screw point capture is shown according to one embodiment. Manually placed screws inherently have no trajectory data and need a method to collect the data required to generate a rod plan. In this system 300, a navigated data collection instrument 302 is configured for relaying point data back to a planning system, such as robot 14. The instrument 302 includes a handle 304 and a probe 306 extending along a central tool axis from a proximal end 308 to a distal end 310. Similar to instrument 212, the navigated instrument 302 includes an array 312 of tracking markers 314 viewable and trackable by a navigation system, such as robot 14. The navigated array 312 may include tracking fiducials 314 arranged in a specific pattern that can be tracked in 3D space by a camera associated with the navigation system, such as robot 14. The instrument 302 may include swappable probes 306 having a distal end configured to precisely match a screw head or tulip head 316. When the screw head 316 is in the proper orientation, the user may actuate a mechanism 318, mechanical or electrical, that reveals a fiducial 320 to the camera to indicate the position of the captured point. In this manner, data on the locations and positions of the manually placed screws may be collected to generate the rod bending plan.

The advantages of an automatic intra-operative rod bending system may include one or more of the following: (1) verifying screw placement locations; (2) precisely bending a sterile rod to plan; (3) adjusting the captured plan to drive correction; (4) recalling and reproducing bent rods; (5) reducing notching and rod defects; (6) decreasing operative time; (7) lowering the skill barrier and learning curve; and (8) offloading rod bending from the surgeon's tasks.

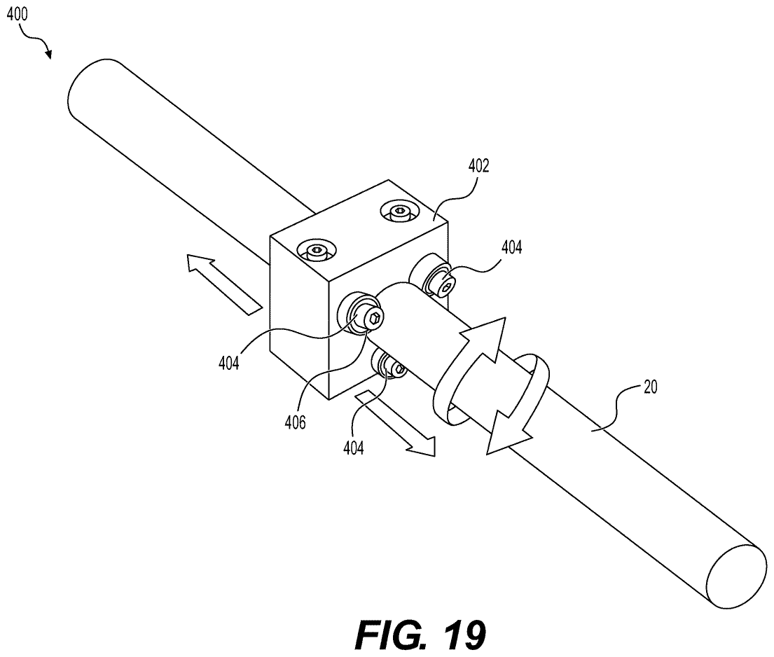

Turning now to FIGS. 19-21, a dual thread-less feed system 400 for feeding the spinal rod 20 for rod bending is shown according to one embodiment. In this embodiment, feeding system 400 is configured to control linear translation and roll angle of the spinal rod 20. The feeding system 400 may include a bearing block 402 and a plurality of bearings or rollers 404 housed therein. The bearing block 402 defines a through opening 406 sized and dimensioned to receive the shaft of spinal rod 20 therethrough. Opening 406 and rod 20 are coaxially aligned along central axis 408. Each of the rollers 404 may have a cylindrical body configured to rotate about a central axis 410. In one embodiment, three rollers 404 may be placed around the spinal rod 20. The rollers 404 are clamped onto the shaft of rod 20 by bearing block 402.

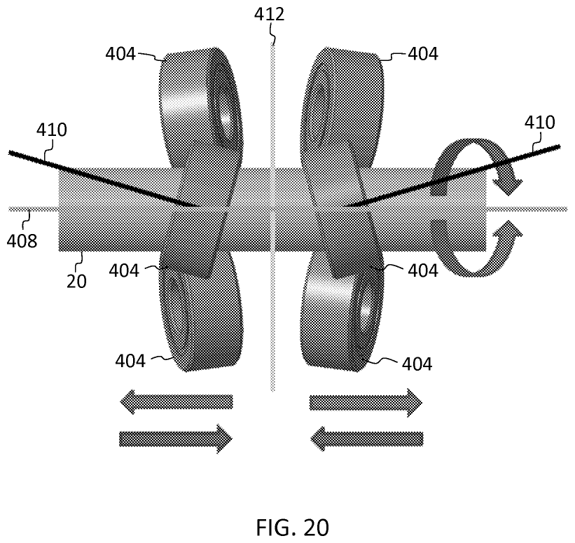

With further emphasis on FIG. 20, the rollers 404 are shown engaged with rod 20 with the bearing block 402 omitted for clarity. The rollers 404 may be mounted such that the central axis 410 of each roller 404 is angled relative to the central axis 408 of the shaft 20. When the shaft 20 is rotated, the bearing 404 rolls on the shaft 20, imparting a thrust load along the axis 408 of the shaft 20 on bearing block 402. With the bearing block 402 constrained such that it cannot rotate, the bearing block 402 moves along the axis 408 of the shaft 20. Counter clockwise shaft rotation gives positive linear motion and clockwise shaft rotation gives negative linear motion. Mirroring the angle of the rollers 404 relative to the central axis 408 across a mirror plane 412, reverses the linear motion when the shaft 20 is rotated. When the bearing block 402 is constrained along the central axis 408, rotating the bearing block 402 rotates the shaft 20. If the shaft 20 is fixed rotationally, the bearing block 402 generates a thrust load along the central axis 408. The advantage of this rod feeding mechanism 420 include minimal to no backlash rod motion, not limited by rod length, coupled feed and roll motion, and thin profile along the central axis 408.

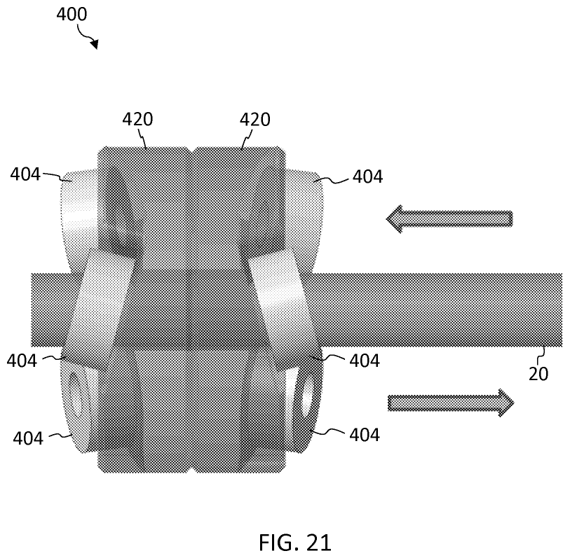

With further emphasis on FIG. 21, a pair of mirrored bearing blocks 402 include rollers 404 that may be rotated independently. As shown in the input table, the inputs may include clockwise and counter clockwise rotation.

Input

| | |
|---|---|
| +θ | counter clockwise rotation |
| −θ | clockwise rotation |

As shown in the differential output table, the shaft's translation along and rotation around the central axis 408 may be controlled.

| Differential | | Block 2 | |
|---|---|---|---|
| Output | | +θ | −θ |
| Block | +θ | +θ | −X |
| 1 | −θ | +X | −θ |

Thus, varying the rate between the two inputs produces combined linear and rotational motion.

Turning now to FIG. 22, a radially adjustable feed system 420 for feeding the spinal rod 20 for rod bending is shown according to one embodiment. In some instances, thread-less screw applications may be designed to work with a single diameter shaft. The bearing block configurations may be based on clamp collars with specific bearing spacing for a given shaft. In some instances, static bearing blocks may be impractical because they require a separate bearing block for each rod size. In this embodiment, the bearings or rollers 404 are mounted on slide blocks 422. The rollers 404 and slide blocks 422 are retained within bearing block 424. In this instance, bearing block 424 may be a circular object, such as disc, cylinder, or wheel. The bearing block 424 defines a cavity 426 for retaining the rod 20, rollers 404, and slide blocks 422. The cavity 426 may be define a tri-lobe cavity with three equally spaced openings. The rod 20 is positioned through the center of the block 424 and each set of roller 404 and slide block 422 are housed within the respective lobes of the cavity. The rollers 404 are mounted on slide blocks 422 that can slide radially within bearing block 424. Each of the slide blocks 422 may be spring loaded with a spring 428. The spring loaded slide blocks 422 allows for radial variability to accommodate multiple rod diameters and apply constant force on the rod 20 needed to generate the force vectors. The advantage of radially adjustable rollers 404 is that the system 420 is configured to accommodate multiple rod diameters and allows for clamp force tuning.

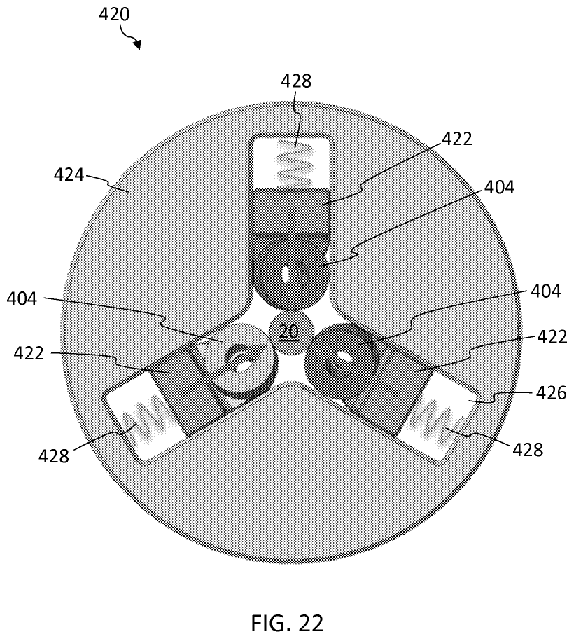

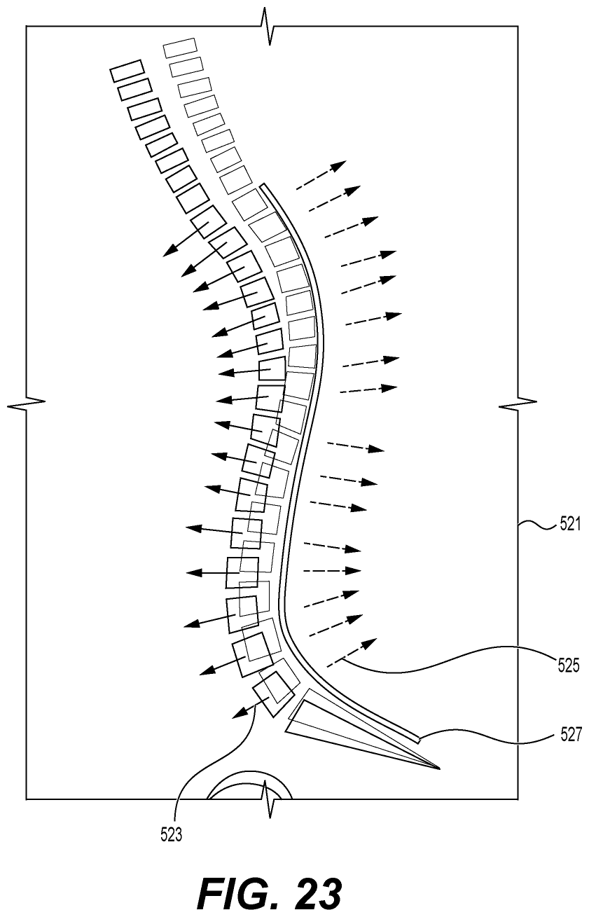

Turning now to FIGS. 23-29, the rod bending workflows described previously with reference to FIGS. 14-17 may include additional data-driven programming to achieve associated benefits for pre-operative rod plans, intra operative rod plans, or in situ or manual procedures. Such associated benefits applicable to a variety of surgical plans associated with a patient's spine and spinal correction, whether accomplished through a robotic systems or navigated instruments.

In certain implementations of spine surgery plans, a particular patient's spine stiffness may be factored into the rod-and-screw construct of the surgical plan, including the rod bending plan. For estimating the correction needed in a patient with spinal deformity, a computerized graphical user interface, shown schematically at 521 in FIG. 23 allows the surgeon to simulate, through suitable computer programming of the system, bone repositioning in a 3D geometric model or 3D scan volume of the patient's spine. The representation corresponding to individual vertebra can be moved as a rigid body relative to other vertebrae by the system receiving suitable user input, such as dragging its image using a mouse or touch screen, thereby updating the surgical plan. Alternately, the workflows implemented by the system program would permit an ideal curvature to be estimated. In one version, based on the system having access to data corresponding to a typical healthy spine curvature, the geometric model or 3D scan volume would be modified so that it matches the ideal curvature.

The foregoing corrections may be performed without consideration of forces and, as such, would be intended only to estimate the desired position of the spine. With the system storing or displaying the spine moved to the desired orientation, screws can be manually or automatically planned and overlaid on the medical image or 3D geometric model.

The system may include suitable programming and associated data to factor in a patient's anatomical spine stiffness into the spinal correction and associated spinal surgical or rod bending plan. For example, in certain situations, without factoring in such data, if a bent rod were planned for connecting the screw heads based on the desired curvature simulated, the rod may not completely move the vertebrae to their targets without further in-situ or intra-operative adjustments, once attached to the screw heads because the spine itself would be applying a counteracting force against the rod, acting to un-bend the rod. The computerized system programmed as described herein factors in the effect of a patient's spine stiffness or counterforces shown schematically at 523 in FIG. 23 on rod forces 525 associated with the contemplated rod curvature or bend of rod 527.

As such, a desired rod curvature may be determined by the system program that is bent beyond a previously determined value associated with what was believed to be an optimized desired spine curvature, so that when deployed, after the patient's spinal system comes to equilibrium, the desired spine curvature or spine correction is achieved.

The programmed system and patient-associated spine data thus creates a biokinetic model that accounts for the effects of the stiffness of the patient spine on the stiffness of a proposed screw-rod construct. The system programming of the model would take as inputs the rod and screw parameters (diameter and material) and parameters that affect the stiffness of the spine connective tissues—especially ligaments and disks, which control the static curvature of the spine—as well as other spine stiffness parameters, such as a patient's age, gender, bone quality, radiographic status of intervertebral disks, smoking history, and ethnicity. The biokinetic model generated by the system programming disclosed herein would be configured to have modeled structures such as vertebral bodies and intervertebral disks, as well as ligaments and one or more muscle connections. After determining such configuration, system programming associated with the biokinetic model would provide an improved estimate of a rod configuration for a corresponding rod plan to reach the desired spine curvature after the rod and spine come to equilibrium.

After running the simulation, the surgeon may find that the required amount of bending of the rod makes it unwieldy and difficult to deploy. Surgical resections of the spine such as osteotomies, discectomies and ligamentous resections can reduce the stiffness that the spine would exert against the screw-rod construct. System programming associated with the biokinetic model may be executed in response to surgeon input to estimate the change in spine stiffness caused by each of these surgical procedures and estimate the new necessary rod curvature to achieve the desired spine curvature after these stiffness-reducing procedures are implemented. The surgeon may selectively input or otherwise cause computer instructions to be executed to simulate any number of surgical resections to find a set of procedures that would allow the correction of deformity with a rod that has an extent of bending that the surgeon considers reasonable.

Alternately, an algorithm could explore different plans for surgical resections and corresponding bent rods that would be needed for correcting the deformity, and the system programming could provide a set of suggested surgical plans or determine one plan to best meet the surgeon's criteria. Different criteria could be, for example, (A) the least amount of rod bending, (B) the least number of surgical resections, (C) surgical resections targeted at achieving a rod with exactly X % of overbend relative to the target spine curvature (where X=20%, for example).

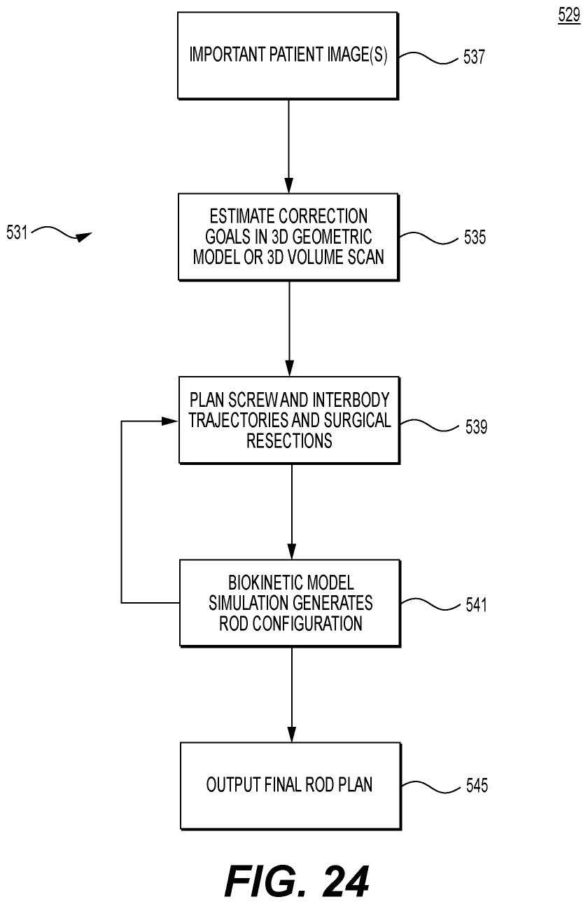

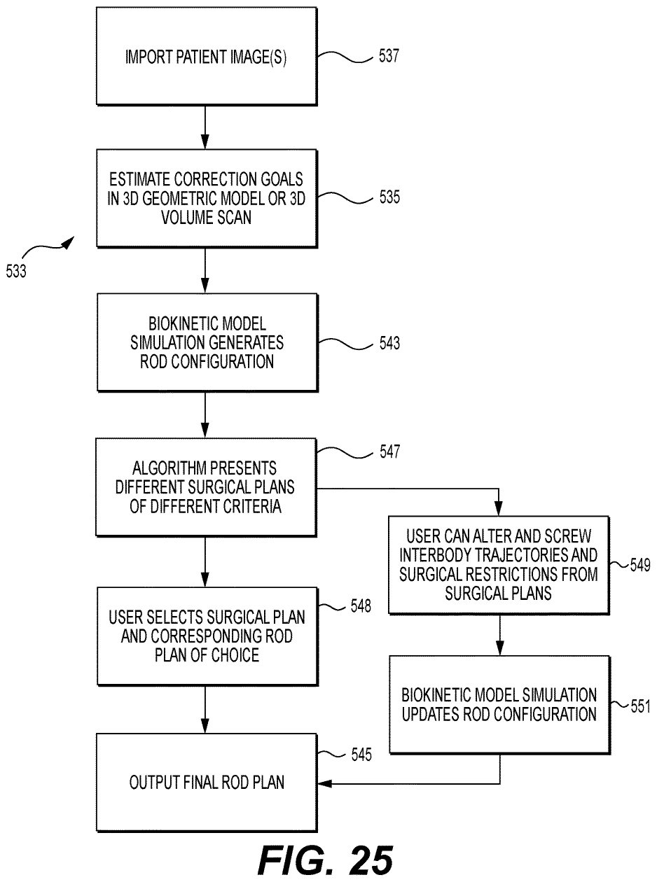

Referring more particularly to FIGS. 24 and 25, a computer-implemented system 529 for generating a spine surgery plan, such as a rod plan and associated rod bending, has programming and associated instructions which, when executed, permit users, such as surgeons to undertake a variety of workflows 531, 533, in connection with pre-operative, intra-operative and manual spine surgery plans, including associated rod bending. Suitable programming generates a graphical representation of a patient spine requiring a spinal correction (535), based on patient spine data accessible to the system (537). As part of the workflow, the system receives input corresponding to repositioning of vertebrae of the patent spine to achieve the spinal correction, associates a set of screws with the spinal correction, and generates a preliminary rod-and-screw construct connecting the set of screws to one or more rods, the parameters of which rods are accessible to the system (539).

System programming performs a biokinetic model simulation, either as part of a predominantly manual rod plan (541) or as part of a more system-driven, adaptive or automatic plan (543). Such simulation includes a variety of executable instructions and corresponding processing, including determining a rod-and-screw stiffness for the preliminary rod-and-screw construct. Such stiffness determination may factor in inputted data about the rods, such rod input including rod material and dimension, and may likewise factor in angulation corresponding to the types of screws in the set of screws, such types potentially including monoaxial, uniplanar, and polyaxial. Suitable programming of the biokinetic model simulation may likewise determine a spine stiffness factor which corresponds to a spinal counterforce affecting the proposed spinal correction. Such spine stiffness factor may be determined as a function of patient stiffness data, such data, in turn, accounting for static forces generated by spinal connective tissues, such as ligaments, discs, and muscle. Patient stiffness data may also encompass more extended factors, such as patient age, gender, bone quality, radiographic status of intervertebral discs, negative health factors (such as smoking), and, to the extent relevant, ethnicity.

In response to determinations of the stiffness of the rod-and-screw construct by itself, on the one hand, and how the patient's spine stiffness factor may affect the desired spinal curvature or correction (such as by exerting spinal counterforce on the rod-and-screw construct), on the other hand, suitable programming may modify components or configuration of the preliminary rod-and-screw construct to generate an updated rod configuration. Such modifications may relate to any number of variables associated with the rod or rods included in the rod plan, including aspects of a complex three-dimensional configuration proposed for the rod and accomplished by bending. In one suitable programmed implementation, the preliminary rod-and-screw contrast includes a first proposed rod curvature associated with the desired patient spinal curvature outcome, and the modification after processing with the biokinetic model simulation programming determines a second proposed rod curvature based on the spine stiffness factor and the rod-and-screw stiffness to simulate the patient spinal curvature outcome when the spine is at equilibrium.

Depending on the workflow and user preferences, such updated rod configuration may be outputted as the final rod plan (545), or may be associated with further process steps. Such further processing may include executing the programming with different rod-and-screw constructs and different amounts or types of surgical resections (or no resections) (549) to generate and present to the user, such as the surgeon, different surgical plans having different components or meeting different criteria (547), such as minimizing resections or limiting rod bending. Upon presentation of different surgical plans, the user may indicate to the programmed system 529 the plan such user wishes to perform (548) with assistance of the robotic or navigational components of system 529, and suitable instructions may be executed to commence the spinal procedure. The workflow may also involve altering screw and interbody trajectories and surgical resections from any number of proposed rod plans or surgical plans and updating a corresponding rod configuration and rod plan to reflect such alterations (551).

The programmed system may have access to a surgical resection database including stiffness change values corresponding to any number of types of spinal surgical resections, such as osteotomies, discectomies, and ligamentous resections. As such, suitable programming may receive user input correspond to a proposed surgical resection, determine a corresponding one of the stiffness change values associated with the proposed surgical resection, recalculate the spine stiffness factor based on the determined stiffness change value, and determine an additional proposed rod curvature different from preceding determinations.

Referring again to the initial determination of the patient spinal correction to be achieved, input as to the position of vertebrae for the proposed correction may be received relative to the three-dimensional model by any suitable interface and related programming, whether through user-selected displacement of one of the vertebrae relative to another, such as through keyboard commands, entry of displacement, rotation, or other values related to repositioning of one or more vertebrae, through mouse displacement, touchscreen interfaces, and the like.

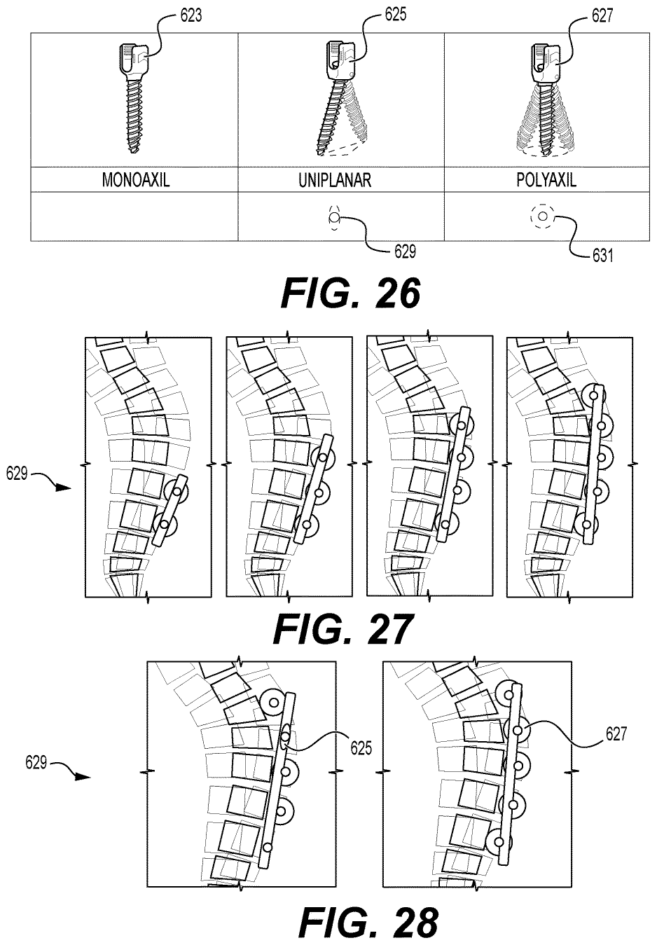

Data-driven approaches to spinal surgical plans, including rod plans and associated rod bending, may alternately or additionally be accomplished by suitable programming for factoring in screw head reachability. Referring more particularly to FIGS. 26-29, a system 621 programmable for bending spinal rods for a patient requiring a spine correction may incorporate screw angulation, screw trajectories, and other screw parameters in workflows for a user, such as a surgeon, in generating rod plans. In one implementation, factoring in screw parameters in rod planning may optimize certain aspects of the surgical plan or the associated rod-and-screw construct, such as minimizing rod bending or other undesirable forces affecting the desired spine correction or patient outcome, or maximizing flexibility for surgeon to adapt to patient particularities in real time intra-operatively or in-situ. Suitable programming may be executed to generate a proposed or simulation rod plan, such as in real time, as the user is inputting data corresponding to a proposed set of screws.

The system may include or otherwise access data for monoaxial screws 623, uniplanar screws 625, and polyaxial screws 627, each of which type generally have different geometric or other structural variables that may be factored in to rod planning. As best seen in FIG. 26, monoaxial screws have no angulation, uniplanar screws have angulation 629 across the plane created by the screw axis and rod axis, and polyaxial screws have angulation 631 in a cone relative to the screw axis.

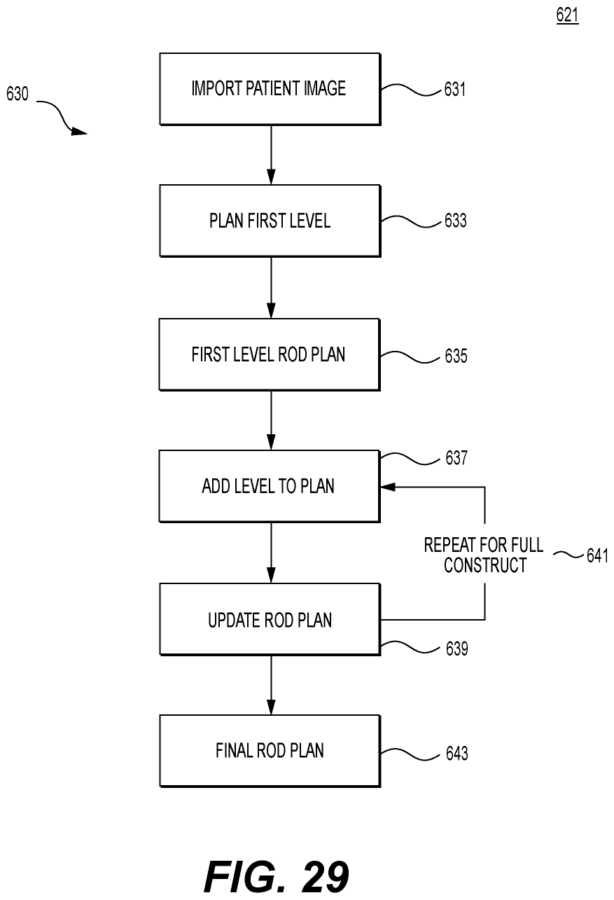

In one exemplary workflow 630 shown in FIG. 29, system 621 accesses patient spine data (631), such as patient images, shown schematically as images of a graphical user interface 629 in FIGS. 27 and 28. The system 621 includes programming to factor in the foregoing screw properties to generate a rod plan which identifies one or more screw types which pass or optimize screw reachability while also minimizing rod bends. In one possible workflow, as the user places screws (633), the simulation generates a rod plan between the screw heads (635). As just one illustrative case, if a screw, such as a uniplanar screw 625 is placed in an unreachable location (FIG. 28), the user can adjust the screw trajectories or substitute a polyaxial screw 627 (workflow step 637), and suitable programming generates a simulated rod update (639), such as adaptively or in real-time. Any of a variety of the foregoing adjustments to or substitutions for screw types or other screw parameters may be inputted in any appropriate sequence and generate updated rod plans in real time or otherwise, and may be repeated by the user as appropriate for the contemplated rod plan, rod-and-screw construct, and associated surgical or rod plan (641). The corresponding workflow may be ended at any point to generate a final rod plan (643).

Alternately, the screw head reachability can be considered in predicting the optimal rod curvature in workflows incorporating spine stiffness factors, such as those examples in FIGS. 24 and 25, using the screw reachable limits to decrease the overall amount of rod bend needed.

In one further potential workflow based on workflow 630, screw reachability may be used to maintain options or surgical procedure flexibility associated with the surgical or rod plan, such as in conjunction with allowing final tightening to occur after the rod is seated in the screw head and the head not yet fully tightened. In such situation, if a rod may otherwise become over bent, it may be difficult to force the spine into a position that allows the screw heads to engage with the bent rod. However, suitable programming may factor in reachability and thus generate one or more adjustable head locations to address such potential difficulties, such as generating available intermediate positions of the rod, where it is engaged but not fully in the contemplated position of the proposed spine correction. In such workflow, once all the screw heads have been engaged with the rod, the surgeon can then apply isolated counter-force to the spine across individual motion segments, causing the screw heads to toggle and slide relative to the rod, and while holding this counter-force, lock those segments into the desired final rigid orientation. The simulation generated by the programmed system 621 can thus exploit screw head variability effectively and ensure that screw heads are positioned where their toggle-ability gives the most flexibility to the surgeon and corresponding benefit in this way.

Workflow 630 and other workflows associated with programmed system 621 may involve further data-driven steps related to screw reachability. In such related implementations, system 621 includes suitable programming to calculate a rod curvature associated with a set of screws determined to pass reachability. However, the system compares such calculated rod curvature to a predetermined rod curvature value, determines if the calculated rod curvature exceeds the predetermined rod curvature value, and receives input corresponding to a substitute screw. The suitably programmed system may update the rod curvature calculation to reflect effect of the substitute screw thereon, and re-determine whether the updated rod curvature does not exceed the predetermined rod curvature. In this way, for example, if a determination is made that the spinal correction should not have a rod bend exceeding a certain arc or other relevant configuration, the system can access such data, either from a database or through user input, and screw adjustments may be made to stay within the predetermined limits for the rod. In one variation, suitable programming, in response to a determination of exceeding the predetermined rod curvature, an output is generated by the system identifying to the user at least one alternative screw having angulation or other screw parameters which avoid a determination of exceeding the predetermined rod curvature.

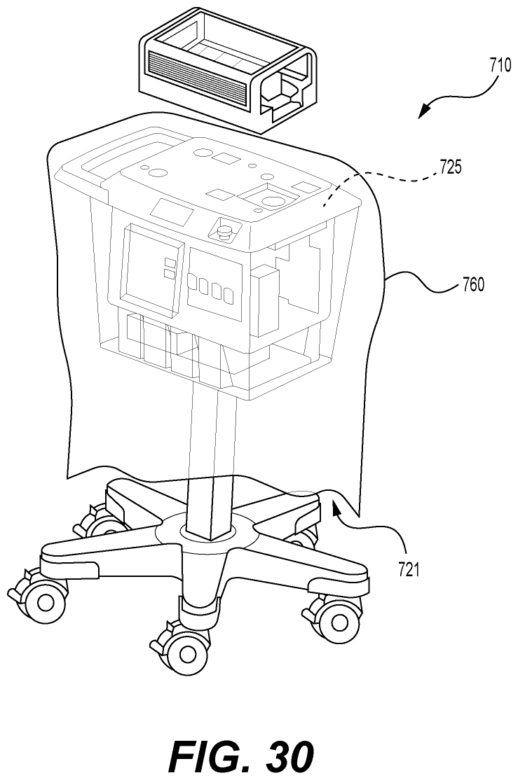

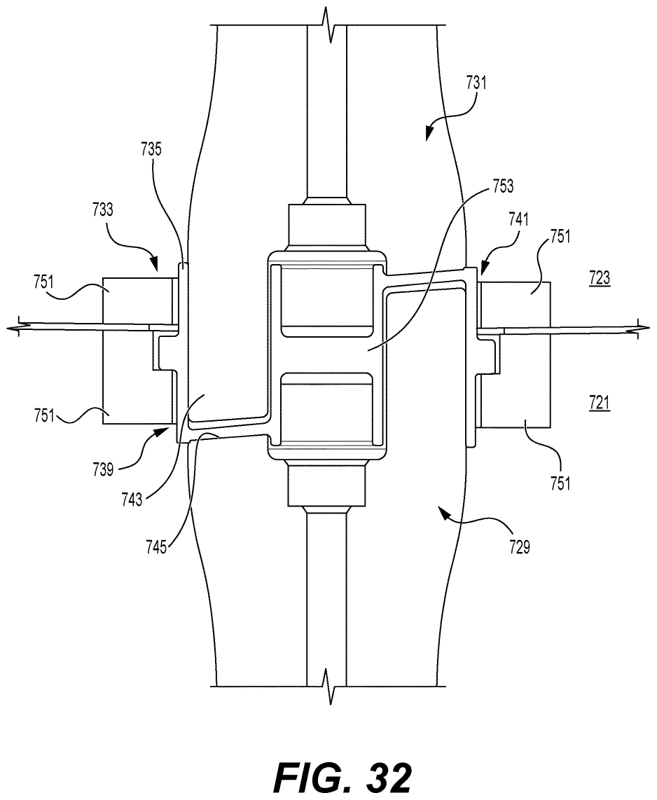

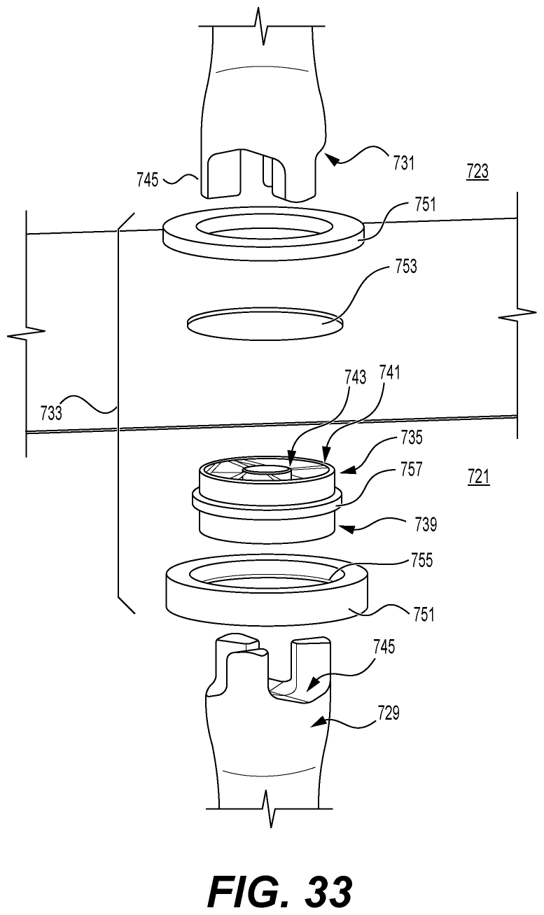

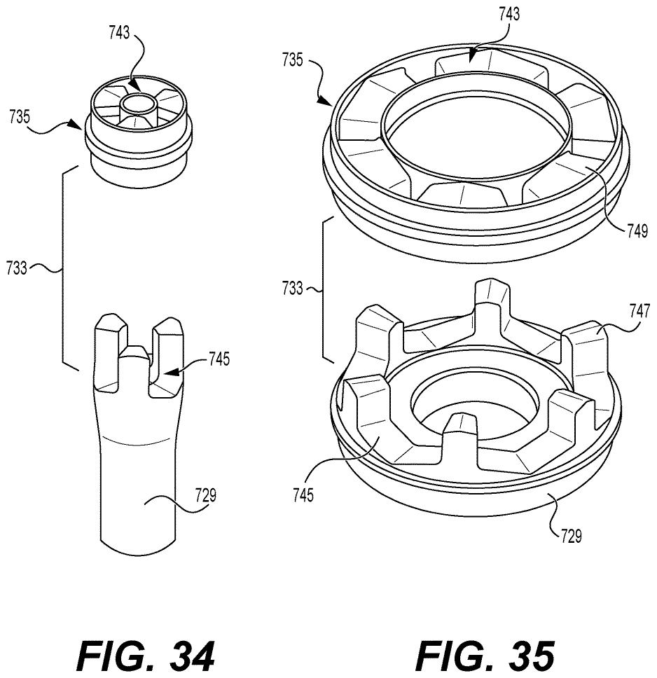

Referring now to FIGS. 30-37, the rod bending system 10 described herein (FIGS. 1-4), may include certain components, features or configurations associated with sterile drape 60 or similar sterile barrier, and which facilitate the transfer of powered operations from the non-sterile side to the sterile side, such as through a non-sterile implementation of coupling member 48 and a sterile implementation of actuated coupling member 50. In the implementations of FIGS. 30-37, a system 710 for bending a spinal rod 20 (FIG. 1) includes a sterile barrier 760, such as a sterile drape, having opposite surfaces and extending sufficiently to define a non-sterile side, region or zone (collectively corresponding to reference 721), and a sterile side, region or zone (collectively, 723). Non-sterile side 721 may be associated with bender box 16 (FIG. 1) or, in the implementations illustrated in FIGS. 30-37, a motion table 725. Sterile side 723 may be associated with robot 14 and bender assembly 18 (FIG. 1), or, in the implementations illustrated in FIGS. 30-37, a sterile cartridge 727, as well as a robot, robot arm, or corresponding end effector associated therewith. In certain implementations, the motion table 725 or bender box 16 comprise an actuated, non-sterile coupling 729, on the non-sterile side 721 which is attachable to a sterile coupling 731, which may comprise bending mandrel 64 (FIG. 5). Attachment between the non-sterile and sterile couplings 729, 731 occurs by means of a mechanical connection 733 through sterile barrier 760.

Mechanical connection 733 may assume a variety of configurations, depending on the nature of the power, motion, or operation to be transferred from motion table 725 or other powered components on non-sterile side 721 to the sterile cartridge 727, bender assembly 18, or bending mandrel 64, or other sterile portions accessible to robotic arm or its end effector. In its various configurations of FIGS. 30-37, mechanical connection 733 is operatively interconnected to the non-sterile coupling and the sterile coupling, which includes the ability to transfer power or motion across sterile barrier 760 without significant compromise between sterile and non-sterile sides 723, 721, and without significant disruption to or displacement of sterile barrier 760.

In certain implementations, mechanical connection 733 comprises a sterile floating coupling 735 rotatably received in a coupling housing 737. Coupling housing 737 is suitably secured, such as by heat bonding or similar fixative processes, to sterile barrier 760, and extends between the opposite surface thereof to define opposite, non-sterile coupling side 739 and sterile coupling side 741.

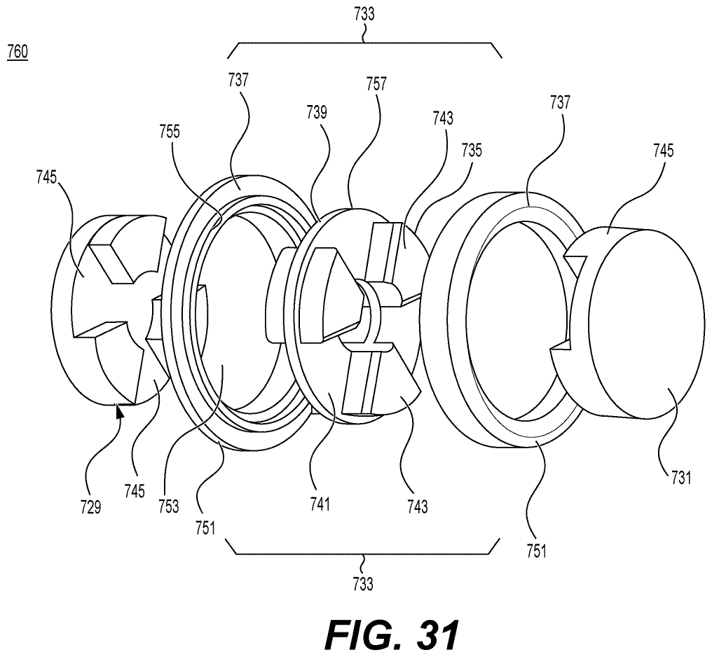

One or both sides 739, 741 of floating coupling 735 may include keyed features 743, such as teeth or flanges, which features may be releasably connected to mating portions 745 of opposing surfaces of non-sterile coupling 729 or sterile coupling 731. As seen in FIGS. 30-34, the arrangement of keys 743 and mating portions 745, such as slots, may be located in any number of configurations on opposing surfaces of the implementations and configurations of floating coupling 735 and the non-sterile and sterile couplings 729, 731, so long as there is operative interconnection to transfer motion, such as rotation, and associated power, across the sterile barrier 760. The implementations illustrated in FIGS. 31-34 make use of three outwardly extending tabs separated by three grooves, the grooves and tabs extending circumferentially through 60 degrees of arc. As shown in the implementations illustrated by FIG. 35, suitable releasable engagement may be accomplished by mating or intermeshing teeth 747 and grooves 749, radially disposed about the opposing surfaces of the non-sterile coupling 729 and floating coupling 749. Still further implementations may further vary the scale, specific geometry overall or of keyed and mating features of floating coupling to fit the application. In certain exemplary implementations, lower load applications can use smaller or fewer keys 743 and mating portions 745, or other complementary drive features, whereas higher load applications could necessitate larger or more numerous keys, mating portions or drive features. The implementation of FIGS. 31-34 show three keys or, alternatively, lobes, and the relatively more complex geometry of FIG. 35 show a six key or lobe application. The surfaces of non-sterile and sterile couplings 729, 731 (FIGS. 31-34) which oppose floating coupling 735 may be scaled or otherwise configured to mate accordingly. Floating couplings may have geometric features to assist with alignment relative to coupling housing 737, or the opposing surfaces of non-sterile and sterile couplings 729, 731.

In certain implementations, keys 743 and mating portions 745 may be releasably interconnected so that barrier 760 can be readily separated from engagement with couplings on the non-sterile and sterile sides, or releasable relative to the bender box and the bender assembly.

Implementations of coupling housing 737 may vary depending on power and motion requirements, as well as configurations of opposing non-sterile and sterile couplings. In the illustrated implementations of FIGS. 31-33, coupling housing comprises a pair of mating rings 751 connected to surrounding portions of the sterile and non-sterile surfaces of sterile barrier 760. Rings 751, in turn, surround an aperture 753, sized and configured relative to coupling housing 737 so that floating coupling 735 is rotatably mounted relative thereto. Rings 751 have opposing surfaces engaged in a substantially airtight seal to minimize air passing between the sterile and non-sterile sides 723, 721 defined by sterile barrier 760. Housing 737 may include a circumferential structure 755 around aperture 753, such as a flange or channel, configured to engage edge portions 757 of sterile coupling 731 and rotatable relative thereto.

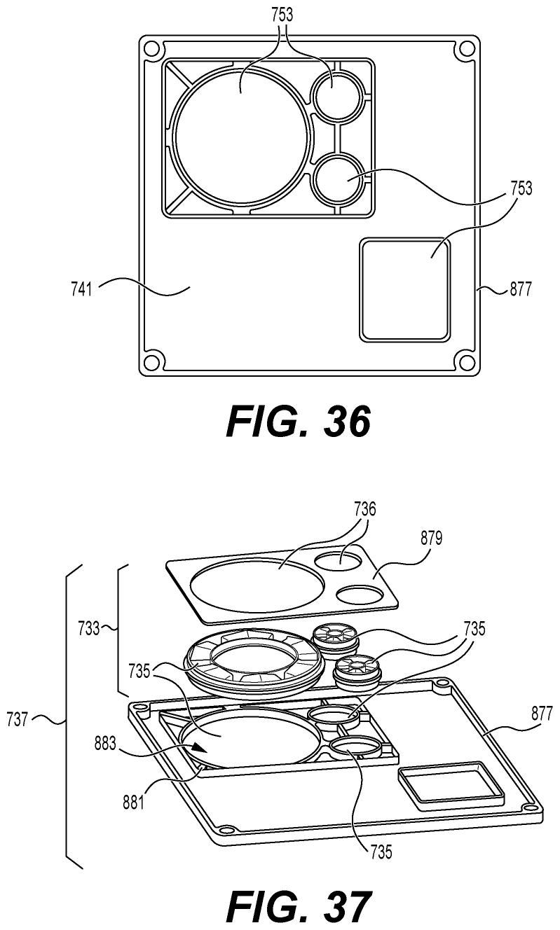

As seen in particular in FIGS. 36 and 37, another implementation of a coupling housing 837 may have one or more rectangular housing components 877, 879, which define apertures 753 at predetermined locations for a plurality of floating couplings 735. Coupling housing 837 may be selectively secured to sterile barrier 760 at a location to correspond to top plate 42 of bender box 16 (FIG. 2), or top surface of motion table 725. Sterile drape 760 equipped with housing 837 may be located in operative proximity between the upper surface of motion table 725 and sterile cartridge 727 to transmit rotation therebetween. Sterile cartridge 727 may be removable for sterilization separate from other components and may likes have portions for receiving sterile spinal rod 20 (FIG. 1) therein and applying at least one of a bending force or a cutting force to such spinal rod 20.

Housing component 877 in this implementation is located below sterile barrier 760 and has a subportion 881 with inner walls defining a tray 883. Mating housing component 879 is sized to fit into tray 883 in an interference fit, and has at least one aperture 736 defined therein and located to communicate with respective one or more apertures 735 located in tray 883 of housing component 877. Sterile barrier 760 is interposed between lower surface of mating housing component 879 and component 877. In this way, sterile tray portions are defined above component 879, such that floating coupling 735 received in aperture 735 defined in tray 883 has its non-sterile side below tray 883 and its sterile side above it.

Sterile drape 760 and its one or more floating couplings 735 may be used in any number of robotic or powered surgical settings to isolate non-sterile powered mechanical components actuatable by such systems, such as those robotic systems used to perform robotic-assisted spinal operations.

Methods of use of sterile drape 760 and its mechanical connections 733 are apparent from the foregoing description. In one exemplary method of use, one places sterile barrier 760 between the non-sterile powered mechanical components associated with a contemplated spinal procedure of the operation, on the one hand, and a sterile location accessible to a movable arm of the robotic surgical system located in the sterile field, thereby defining non-sterile and sterile regions of the robotic surgery system. Thereafter, by virtue of the sterile barrier having at least on floating coupling mounted thereto and extending between non-sterile and sterile sides of the sterile barrier, the floating coupling may be moved by the powered mechanical components without inducing movement of the sterile barrier. In other uses, one can position the floating coupling in operative proximity to a predetermined one of the powered mechanical components, and interconnect the predetermined one of the powered mechanical components and the sterile location through the floating coupling to permit the robotic assisted procedure on the sterile side in response to actuation of the predetermined one of the non-sterile mechanical components. In this way, the sterile region is isolated from the non-sterile mechanical components during the robotic assisted procedure.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A method for isolating non-sterile powered mechanical components actuatable by a robotic surgery system to perform a robotic-assisted spinal operation on a patient in a sterile field, the method comprising:
placing a sterile barrier between the non-sterile powered mechanical components associated with a contemplated procedure of the operation, and a sterile location accessible to a movable arm of the robotic surgical system located in the sterile field, thereby defining non-sterile and sterile regions of the robotic surgery system, the sterile barrier having at least on floating coupling mounted thereto and extending between non-sterile and sterile sides of the sterile barrier, the floating coupling mounted to be movable without inducing movement of the sterile barrier;
positioning the floating coupling in operative proximity to a predetermined one of the powered mechanical components; and
interconnecting the predetermined one of the powered mechanical components and the sterile location through the floating coupling to permit the robotic assisted procedure on the sterile side in response to actuation of the predetermined one of the non-sterile mechanical components, whereby the sterile region is isolated from the non-sterile mechanical components during the robotic assisted procedure.

2. The method of claim 1, wherein the step of interconnecting the predetermine one of the mechanical components comprises connecting a non-sterile side of the floating coupling to an actuated coupling member mounted to at least one of a top surface of a motion table and a bender box, and connecting a sterile side of the floating coupling to at least one of a replaceable sterile cartridge and a bender assembly.

3. The method of claim 2, wherein the step of interconnecting the sterile side of the floating coupling includes rotatably connecting the floating coupling to a sterile bending mandrel mounted on the at least one of the sterile cartridge and the bender assembly.

4. The method of claim 3, wherein the step of rotatably connecting the floating coupling comprises releasably connecting surfaces of the floating coupling to first opposing surfaces of the actuated coupling on the non-sterile side of the floating coupling and to second opposing surfaces of the bending mandrel to form respective pairs of mating surfaces.

5. The method of claim 1, further including the step of positioning a plurality of the floating couplings in operative proximity to corresponding predetermined mechanical components to selectively perform a corresponding plurality of robotic-assisted procedures.
